(19) United States
(12) Patent Application Publication
(10) Pub. No.: US 2021/0180084 A1
(43) Pub. Date: Jun. 17, 2021

(54) GENE THERAPY VECTORS COMPRISING S/MAR SEQUENCES

(71) Applicant: VIVET THERAPEUTICS, Paris (FR)

(72) Inventors: Rafael ALDABE, Navarra (ES); Aquilino LANTERO, Navarra (ES); Gloria GONZALEZ ASEGUINOLAZA, Navarra (ES)

(73) Assignee: VIVET THERAPEUTICS, Paris (FR)

(21) Appl. No.: 17/055,673

(22) PCT Filed: May 14, 2019
(86) PCT No.: PCT/EP2019/062293

§ 371 Date: Nov. 16, 2020

(30) Foreign Application Priority Data
May 14, 2018 (EP) 18305591.2

(51) Int. Cl. C12N 15/86; C12N 9/00; A61P 1/16

(52) U.S. Cl. CPC C12N 15/86; C12N 9/93; C12Y 603/04005; A61P 1/16; C12N 2830/50; C12N 2750/14171; C12N 2750/14143; C12N 2750/14123; C12N 2750/14151; C12N 2830/46; C12N 2750/14122

(57) ABSTRACT

A field of gene therapy and engineering of viral vectors for use in gene therapy. More specifically, it is disclosed herein adeno-associated virus vectors and expression cassettes comprising S/MAR sequences of c-Myc or IFN-β for the treatment of liver diseases, notably in neonates.

### TECHNICAL FIELD

The present disclosure relates to the field of gene therapy and engineering of viral vectors for use in gene therapy. More specifically, it is disclosed herein adeno-associated virus vectors and expression cassettes comprising S/MAR sequences of c-Myc or IFN-β for the treatment of liver diseases, notably in neonates or infants.

### BACKGROUND

Inherited metabolic diseases comprise a large class of hereditary genetic diseases that involve disorders of the metabolism. Monogenic metabolic liver diseases are a significant subgroup of these diseases caused by defects of single genes encoding proteins (often enzymes) that allow the regulation of complex metabolic pathways, or circulating proteins, mainly produced by the liver. These defects, reduce the ability to synthesize essential compounds and/or generate an accumulation of toxic intermediate metabolites that cannot be processed inducing toxicity in different organs. Current treatment is often limited to supportive measures and may entail significant adverse effects and impairment in the quality of life. Up to now, liver replacement therapies remain the only established curative treatment for many monogenic metabolic liver diseases due to the ability to restore the defective pathway. However, the shortage of donors, the associated mortality/morbidity and the need for immunosuppression, often limit this option to severely affected patients. Given the severity and prevalence of these diseases, great efforts have been made in recent decades to develop more effective strategies to treat the complications associated with these diseases, prolong survival and improve the quality of life. However, for most diseases there are no effective therapies.

In the past decade, gene therapy, by providing additional functional gene copies, has emerged as a promising alternative to transplantation in monogenic metabolic liver diseases. Gene therapy, through the use of vectors based on adeno-associated viruses (AAV), represents today one of the most promising therapeutic strategies for these diseases. AAV are non-integrative vectors used in gene therapy that are maintained episomally in the cells and have demonstrated an excellent safety profile in different clinical trials. Many of clinical trials have been performed using AAV viral vectors for monogenic disorders, with the first human AAV injection performed almost 25 years ago (Flotte et al., 1996). Examples of clinical efficacy with AAV include data from trials with hemophilia B (Nathwani et al., 2011; Nathwani et al., 2014), alpha 1 antitrypsin (Flotte et al., 2011; Cideciyan et al., 2013) and Leber congenital amaurosis (Bainbridge et al., 2015). However, as observed in other previous trials, to achieve therapeutically relevant transgene expression high vector doses were required (Nathwani et al, 2011). For efficiency of a gene therapy, maintenance of viral genomes is therefore also required.

Moreover, a large number of these monogenetic metabolic liver diseases begin to affect the patients in early stages of life, even in the neonatal period, and many of them produce devastating consequences on the children development. These early metabolic diseases require soon therapeutic intervention. Therefore, it is essential to have effective early treatments.

Neonatal gene transfer has the advantages of achieving therapeutic effects before disease manifestation, a low vector requirement and high vector-to-cell ratio, and a relatively immature immune system. However, one of the major limitations with early AAV therapy is the loss of viral genomes during liver growth and the maturation process of hepatocytes. After AAV treatment, the liver of neonates continues growing and as the used vectors have no potential to replicate or integrate, the percentage of cells with the therapeutic vector decreases over time. This means that once the liver growth ends, there is a very low number of hepatocytes that conserve the therapeutic vector (Sharon et al., 2008; Wang et al., 2012). There is thus a need for AAV vectors resistant to this phenomenon that would allow to address the treatment of hepatic metabolic diseases at some very early stages in the life of patients.

In the genome of eukaryotic cells exist sequences in their chromosomes that interact with the nuclear matrix and mediate structural organization of the chromatin into loops or domains within the nucleus. These sequences are known as Scaffold/Matrix Attachment Region (hereafter referred as “S/MAR”) and play an important role in chromatin organization, transcription and replication (Henry et al., 2004, Hagedorn et al., 2011). These abilities of S/MAR elements have led to investigate their role in different non-integrating expression cassettes to assist in the establishment of episomes over cell division. In non-viral plasmids, S/MAR elements are used to establish long-term gene expression through the interaction with the nuclear matrix maintaining episomal transgene expression over hundreds of cell generations after an initial phase of antibiotic selection. The episomal replication is due to a stable association with early replication foci by the binding of S/MAR elements to the nuclear matrix protein scaffold attachment factor A (SAF-A) (Piechaczek et al., 2009; Schaarschmidt et al., 2004; Haase et al., 2010). Furthermore, S/MAR elements has been cloned into Non-integrating lentiviral (NIL) vectors establishing long-term episomal vector maintenance in dividing cells, even after numerous rounds of cell division, in different cell lines including primary murine cells without any type of selection (Verghese et al., 2014; Kymäläinen et al., 2014; Jin C et al., 2016; Xu et al., 2016).

Furthermore, the use of Matrix Attachment Region in AAV vectors has also been disclosed in WO 2014/016580. HPRT-F/R S/MAR, ApoB-F/R S/MAR, KSHV-F/R S/MAR and IFN-β-F/R S/MAR are compared, and HPRT-F S/MAR and ApoB-R seems to provide the best hFIX expression, whereas IFN-β-F S/MAR seems to provide no effect on hFIX expression by comparison with the control (see FIGS. 8C and 9B). Moreover, this document only discusses the effect of S/MAR elements for transgene expression in old mice. This document never mentions the effect of S/MAR elements for the maintenance of viral genome, and does not mention either gene therapy in neonates, infants or children.

Likewise, S/MAR elements have been sub-cloned into AAV vectors which are designed not to be integrated into chromosomes and vector genomes persist predominantly as episomes, therefore, can only mediate long-term transgene expression in post-mitotic tissues. It has been observed that when S/MAR elements are sub-cloned into an AAV vector, these elements facilitate episomal long-term persistence of AAV vector genomes and long-term transgene expression in proliferating cells after antibiotic selection (Hagedorn et al., 2017). However, these studies have been carried out on cells in culture and on the livers of adult mice, but no one has evaluated their possible role when the viral vectors are inoculated in neonatal or infant livers where there is a continuous proliferation of hepatocytes.

Therefore, the use of S/MAR elements in AAV vectors has been discussed in prior art. However, these documents only discuss their effect on transgene expression, and not their effect on the maintenance of viral genomes. Moreover, these documents disclose the use of S/MAR elements in AAV vectors for gene therapy in adults, but never mention the use of AAV vectors comprising S/MAR elements for gene therapy in neonates, infant or children, notably for the treatment of liver diseases.

There is thus a need for more potent expression cassettes, optimized vectors (notably AAV vectors) for gene therapy which allow the maintenance of the viral genomes and/or transgene expression. There is also a need for more potent expression cassettes, optimized vectors (notably AAV vectors) for gene therapy in neonates, infants or children, notably for the treatment of liver diseases.

The inventors have thus developed such expression cassettes and vectors. The present invention relies indeed on their in vivo results showing that two specific S/MAR sequences maintain viral genomes at a level higher than control AAV or other tested S/MAR sequences. Inventors have notably shown that the number of viral genomes is higher after treatment with AAV vector comprising the specific IFN-β-F S/MAR or c-Myc-R S/MAR than with the control AAV (i.e. that does not comprises S/MAR elements) or than with AAV comprising other S/MAR elements. Such results show notably the effect of the integration of different S/MAR elements into an AAV vector on the stabilization of AAV genomes in the liver of neonates' mice as episomes giving to the AAV vectors the potential to replicate at the same time than the hepatocytes do when the neonate or infantile liver grows.

### SUMMARY

The present invention relates thus to an expression cassette that comprises:
a transgene, and
a scaffold/matrix attachment region chosen among the nucleic acid sequences of SEQ ID NO:12 or SEQ ID NO:2, or a functional fragment or variant thereof.

In a specific embodiment, which can be combined with other embodiments, the expression cassette further comprises a promoter which initiates transgene expression upon introduction into a host cell. Preferably, the promoter is an alpha-1-antitrypsin promoter, notably of SEQ ID NO:19.

In a specific embodiment, which can be combined with other embodiments, the expression cassette further comprises a 5′ITR and a 3′ITR sequences, preferably a 5′ITR and a 3′ITR sequences of an adeno-associated virus, notably a 5′ITR and a 3′ITR sequences from the AAV2 serotype. In a more preferred embodiment, 5′ITR sequence is of SEQ ID NO:16 or SEQ ID NO:23 and 3′ITR sequence is of SEQ ID NO:17.

In a specific embodiment, which can be combined with other embodiments, the expression cassette further comprises a terminator, such as a polyadenylation signal sequence, notably the bovine growth hormone polyadenylation signal sequence, more preferably of SEQ ID NO:18.

In a preferred embodiment, which can be combined with other embodiments, the transgene encodes argininosuccinate synthase 1. Such transgene is notably represented by SEQ ID NO:20.

In a specific embodiment, which can be combined with other embodiments, the expression cassette further comprises the sequence of a MVM intron, upstream the sequence of the transgene. Such MVM intron is notably represented by SEQ ID NO:21.

In another preferred embodiment, which can be combined with other embodiments, the expression cassette comprises or consists of SEQ ID NO:14 or SEQ ID NO:22.

The present disclosure also relates to a recombinant vector comprising an expression cassette as defined above. Preferably, said vector is an adeno-associated virus vector.

The present disclosure also relates to a host cell comprising an expression cassette as defined above or a vector as defined above.

The present disclosure also relates to a viral particle comprising an expression cassette as defined above or a vector as defined above. Preferably, the viral particle comprises capsid proteins of adeno-associated virus, notably capsid proteins from Anc80 serotype.

The present disclosure also relates to a pharmaceutical composition comprising an expression cassette as defined above, a vector as defined above, a host cell as defined above, or a viral particle as defined above, in combination with one or more pharmaceutical acceptable excipient, diluent or carrier, optionally comprising other active ingredients.

Also disclosed herein is an expression cassette as defined above, a vector as defined above, a host cell as defined above, or a viral particle as defined above, or a pharmaceutical composition as defined above, for use as a medicament.

In an embodiment, the disclosure further relates to the expression cassette as defined above, a vector as defined above, a host cell as defined above, or a viral particle as defined above, or a pharmaceutical composition as defined above, for use in gene therapy in a subject in need thereof. In another embodiment, the disclosure further relates to the expression cassette as defined above, a vector as defined above, a host cell as defined above, or a viral particle as defined above, or a pharmaceutical composition as defined above, for the treatment of diseases with hepatic origin, in a subject in need thereof. Preferably such disease is citrullinemia, notably citrullinemia type 1.

In an embodiment, the expression cassette as defined above, a vector as defined above, a host cell as defined above, or a viral particle as defined above, or a pharmaceutical composition as defined above, is used in neonate, infant, child or adult, preferably in neonate, infant or child, more preferably in neonate or infant.

Also disclosed herein is a process for producing viral particles as defined above, comprising the steps of:
a) culturing a host cell as defined above in a culture medium, and
b) harvesting the viral particles from the cell culture supernatant and/or inside the cells.

The disclosure further relates to the use of an expression cassette as defined above, or a vector as defined above, for the production of viral particles.

The disclosure also relates to a kit comprising an expression cassette as defined above, or a vector as defined above, a host cell as defined above, or a viral particle as defined above, or a pharmaceutical composition as defined above, in one or more containers, optionally further comprising instructions or packaging materials.

### LEGENDS OF THE FIGURES

FIG. 1 represents the different AAV genomes generated carrying the reporter gene green fluorescent protein (GFP). AAT: Alpha-1-antitrypsin Promoter; S/MAR: the different S/MAR elements; BGHpA: Bovine Growth Hormone Polyadenylation Signal; ITR: Inverted Terminal Repeat.

FIG. 2 represents a quantification of viral genomes in the liver of the mice 6 weeks after AAV inoculation determined by qPCR and normalized to GAPDH. Data are mean±SEM. *p<0.05, ssAAV-Anc80-GFP versus ssAAV-Anc80-GFP-S/MAR (two-tailed t test). **p<0.01, ssAAV-Anc80-GFP versus ssAAV-Anc80-GFP-S/MAR (two-tailed t test).

FIG. 3 represents a quantification of viral genome in the liver of the treated mice at weeks 1, 3, 6 and 12 after treatment with AAV vectors determined by qPCR and normalized to GAPDH. Graph show the viral genomes loss in folds between week 1 and week 3 and between week 3 and week 6. Data are mean±SEM.

FIG. 4 represents a quantification of viral genomes present in the liver of the mice between the control vector ssAAV-Anc80-GFP and the selected vectors ssAAV-Anc80-GFP-IFN-F and ssAAV-Anc80-GFP-c-Myc-R at week 1, 3, 6 and 12 after inoculation as well as the statistical analysis. Data are mean±SEM. *p<0.05, ssAAV-Anc80-GFP at week 3 versus ssAAV-Anc80-GFP-S/MAR at week 3 (two-tailed t test). **p<0.01, ssAAV-Anc80-GFP at week 3 versus ssAAV-Anc80-GFP-S/MAR at week 3 (two-tailed t test). ++p<0.01, ssAAV-Anc80-GFP at week 6 versus ssAAV-Anc80-GFP-S/MAR at week 6 (two-tailed t test). #p<0.05, ssAAV-Anc80-GFP at week 12 versus ssAAV-Anc80-GFP-S/MAR at week 12 (two-tailed t test).

FIG. 5: represents a quantification of viral genomes and % of transgenic protein positive area in the liver in mice inoculated with the vectors at 2- and 3-weeks of age. (A) Quantification of viral genomes in the liver of treated mice at 2-weeks of age determined by qPCR and normalized to GAPDH. (B) Quantification of viral genomes in the liver of treated mice at 3-weeks of age determined by qPCR and normalized to GAPDH and C) Quantification of the % of transgenic protein positive area of the liver on the transgene vs. total area. Data are mean±SEM. Unpaired two tailed t-test.

FIG. 6: represents a quantification of viral genomes and % of transgenic protein positive area in the liver of mice inoculated at 3-weeks of age. (A) Quantification of viral genomes in the liver of treated mice at 3 weeks of age determined by qPCR and normalized to GAPDH. (B) Quantification of the % of transgenic protein positive area. Data are presented as mean±SEM. Unpaired two tailed t-test.

FIG. 7: (A) Schematic representation of the two rAAV genomes generated: ASS1+INT and ASS1+INT+IFN-β-Forward. EAlb: Enhancer of Albumin; AAT: alpha-1-antitrypsin Promoter; ASS1: Human arginine succinate synthase; S/MAR: S/MAR element; BGHpA: Bovine Growth Hormone Polyadenylation Signal; ITR: AAV Inverted Terminal Repeat. (B) Quantification of viral genomes in the liver of treated Fold/Fold mice at 3 weeks of age determined by qPCR and normalized to GAPDH. (C) Quantification of ASS1 mRNA levels in the liver of treated mice determined by RT-qPCR and normalized to Histone gene transcription.

### DETAILED DESCRIPTION

### Expression Cassette and Expression Vector

The disclosure first relates to an expression cassette that comprises:
a transgene, and
a scaffold/matrix attachment region chosen among the nucleic acid sequences of SEQ ID NO:12 (c-Myc-R) or SEQ ID NO:2 (IFN-(3-F), or a functional fragment or variant thereof.

As used herein, the term “expression cassette” relates to a construct that contains the necessary regulatory elements for the expression of at least the contained nucleic acid in a cell.

As used herein, the term “transgene” relates to a gene useful for treating diseases by gene therapy in human. The term “transgene” also encompasses the term “therapeutic transgene”. Diseases intended to be treated by the nucleic acid constructs of the disclosure are notably the diseases with hepatic origin. Examples of transgenes for treating diseases with hepatic origin which can be used, include without limitation: ATP7B, ATP7B-T2, ASS1, ABCB11, PAH, FAH, TAT, HPD, MDR-3 and GBA gene coding nucleic acids. Preferably the transgene encodes argininosuccinate synthase 1 (ASS1). More preferably ASS1 is represented by SEQ ID NO:15 and a gene coding for ASS1 is represented by SEQ ID NO:20. Upstream the transgene, a MVM intron of SEQ ID NO:21 can be used.

As used herein, the term “scaffold/matrix attachment” or “S/MAR elements” or “S/MAR regions” relates to regions that play a fundamental role in genome organization and co-ordination of expression from specific gene loci. They do so by serving as anchor points on DNA for the nuclear matrix and thereby place genes in close proximity to the aforementioned nuclear proteins required for gene expression. S/MARs generally consist of AT-rich regions found in the 5′ or 3′ flanking regions of genes, in introns, or at gene breakpoint cluster regions. In an embodiment, S/MAR sequences which are used are represented by SEQ ID NO:12 (c-Myc-R) or SEQ ID NO:2 (IFN-β-F). SEQ ID NO:2 represents S/MAR region from the human interferon-β (IFN-β) gene and SEQ ID NO:12 represents S/MAR region from the c-Myc regulator gene. The letter “F” in IFN-β-F means that the S/MAR region from the human interferon-β has been inserted Forward in expression cassette or vector of the disclosure, whereas the letter “R” in c-Myc-R means that the S/MAR region from the c-Myc regulator gene has been inserted Reverse in expression cassette or vector of the disclosure.

As used herein “functional fragment or variant thereof” refers to a sequence that do not have 100% identity with SEQ ID NO:2 or 12, but which presents the same properties (e.g. the stabilization of AAV genomes in the liver of neonates' mice). Preferably, “functional fragment or variant thereof” represents sequences having at least 80%, preferably 90% and even more preferably 95%, 96%, 97%, 98%, 99% of identity with SEQ ID NO:2 or 12. As used herein, the percentage of identity between the two sequences is a function of the number of identical positions shared by the sequences (i.e. % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two nucleotide amino acid sequences may also be determined using for example algorithms such as the BLASTN program for nucleic acid sequences using as defaults a word length (W) of 11, an expectation (E) of 10, M=5, N=4, and a comparison of both strands.

In an embodiment, which can be combined with other embodiments, an expression cassette as defined above can further comprise a promoter which initiates transgene expression upon introduction into a host cell. Suitable promoters may include as examples the SV40 promoter, alpha-1-antitrypsin promoter, CMV, EF1alpha, PGK, viral long terminal repeats, as well as inducible promoters, such as the Tetracycline inducible system. In a more preferred embodiment, the promoter is an alpha-1-antitrypsin promoter, even more preferably of SEQ ID NO:19.

In another embodiment, which can be combined with other embodiments, the expression cassette of the disclosure further comprises a 5′ITR and a 3′ITR sequences. As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles. Preferably 5′ITR and a 3′ITR of a virus selected from the group consisting of parvoviruses (in particular adeno-associated viruses), adenoviruses, alphaviruses, retroviruses (in particular gamma retroviruses, and lentiviruses), herpesviruses, and SV40; in a preferred embodiment the virus is an adeno-associated virus (AAV), an adenovirus (Ad), or a lentivirus. More preferably 5′ITR and a 3′ITR are sequences of an adeno-associated virus. In specific embodiments, the expression viral vector may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype now known or later discovered. More preferably, a 5′ITR and a 3′ITR sequences from the AAV2 serotype are used. In an even more preferred embodiment, 5′ITR sequence is of SEQ ID NO:16 or 23 and 3′ITR sequence is of SEQ ID NO:17.

In another embodiment, which can be combined with other embodiments, an expression cassette as defined above can further comprise a terminator, such as a polyadenylation signal sequence. Suitable polyadenylation signal sequences can be from β-globin, SV40, bovine growth hormone, but also synthetic polyadenylation sequences. In a more preferably embodiment, the terminator is the bovine growth hormone polyadenylation signal, notably of SEQ ID NO:18.

In a preferred embodiment, an expression cassette as defined above, comprises or consists of SEQ ID NO:14 (if c-Myc-R S/MAR is used) or SEQ ID NO:22 (if IFN-(3-F S/MAR is used).

The disclosure also relates to a recombinant vector comprising an expression cassette as defined above. The term “vector” also encompasses the term “expression vector” or “recombinant expression vector”. A recombinant vector according to the disclosure contains all the necessary means for its expression.

The cassette or vector of the disclosure may further comprise a variety of other functional elements. For example, if the vector is preferably capable of autonomously replicating in the nucleus of the host cell, elements which induce or regulate DNA replication may be required in the recombinant vector. Alternatively, the recombinant vector may be designed such that it integrates into the genome of a host cell, and DNA sequences which favour targeted integration (e.g. by homologous recombination) are envisaged.

The expression viral vector is typically a recombinant nucleic acid construct comprising or consisting of the cassette as defined above. In an embodiment, expression viral vector of the disclosure comprises then a transgene, and a scaffold/matrix attachment region chosen among the nucleic acid sequences of SEQ ID NO:12 (c-Myc-R) or SEQ ID NO:2 (IFN-β-F), or a functional fragment or variant thereof.

In an embodiment, an expression viral vector of the disclosure comprises at least the 5′ITR and 3′ITR of a virus, S/MAR sequences of SEQ ID NO:12 or SEQ ID NO:2 and the transgene.

In one and preferred embodiment, the expression viral vector is an AAV vector. The AAV genome is composed of a linear, single-stranded DNA molecule which contains 4681 bases (Berns and Bohenzky, (1987) Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least two viral proteins are synthesized from the AAV rep gene, Rep 78 and Rep 52, named according to their apparent molecular weight. The AAV cap gene encodes at least three proteins, VP1, VP2 and VP3. For a detailed description of the AAV genome, see, e.g., Muzyczka, N. (1992) Current Topics in Microbiol. And Immunol. 158:97-129. As used herein, the term AAV vector includes a recombinant viral vector comprising at least the 5′ITR and 3′ITR of an AAV viral vector. Therefore, in a more preferred embodiment, an expression viral vector of the disclosure comprises at least the 5′ITR and 3′ITR of a AAV virus, S/MAR sequences of SEQ ID NO:12 or SEQ ID NO:2 and the transgene.

In a more particular embodiment, the disclosure relates to a viral expression vector comprising:
5′ITR and 3′ITR sequences of an AAV2 serotype,
an alpha-1-antitrypsin promoter,
a polyadenylation signal sequence, notably the bovine growth hormone polyadenylation signal,
S/MAR sequences of SEQ ID NO:2 or 12.

In an even more particular embodiment, the disclosure relates to a viral expression vector comprising:
5′ITR and 3′ITR sequences of an AAV2 serotype,
an alpha-1-antitrypsin promoter,
a polyadenylation signal sequence, notably the bovine growth hormone polyadenylation signal,
S/MAR sequences of SEQ ID NO:2 or 12,
the transgene encoding argininosuccinate synthase 1.

In an even more particular embodiment, the disclosure relates to a viral expression vector comprising:
a 5′ITR sequence of SEQ ID NO:16 or 23,
an alpha-1-antitrypsin promoter of SEQ ID NO:19,
a MVM intron sequence of SEQ ID NO:21,
the transgene encoding argininosuccinate synthase 1 represented by SEQ ID NO:20,
S/MAR sequences of SEQ ID NO:2 or 12,
a polyadenylation signal sequence, notably the bovine growth hormone polyadenylation signal of SEQ ID NO:18,
and 3′ITR sequences of SEQ ID NO:17.

In another embodiment, the expression viral vector is an adenoviral expression vector. This adenoviral expression vector can be, in particular, a first-, second-, or third-generation adenovirus [see Adenovirus. Methods and Protocols. Chinon M. and Bosch A. (Eds); third Edition; 2014 Springer], or any other adenoviral vector system already known or later described. In these particular embodiment, the expression viral vector of the disclosure is a “third generation adenovirus”, which may also be referred to as “gutless adenovirus”, “helper-dependent adenovirus (HD-Ad)”, or “high capacity adenovirus (HC-Ad)”. A third generation adenovirus has all viral coding regions removed (gutless); it depends on a helper adenovirus to replicate (helper-dependent); and it can carry and deliver into the host cell up to 36 Kbp inserts of foreign genetic material (high-capacity). A gutless adenovirus keeps the inverted terminal repeats ITRs (5′ and 3′) and the packaging signal (w).

In another embodiment, the expression viral vector of the disclosure comprises or consists of a 5′ITR, a ψ packaging signal, a 3′ITR of a virus, S/MAR sequences of SEQ ID NO:12 or SEQ ID NO:2 and the transgene. As used herein “ψ packaging signal” is a cis-acting nucleotide sequence of the virus genome, which in some viruses (e.g. adenoviruses, lentiviruses . . . ) is essential for the process of packaging the virus genome into the viral capsid during replication. In another embodiment, the expression viral vector comprises a 5′ITR, a ψ packaging signal, and a 3′ITR of an adenovirus of any of the serotypes within any of the classification sub-groups (A-G), S/MAR sequences of SEQ ID NO:12 or SEQ ID NO:2 and the transgene. In a particular embodiment, these 5′ITR, ψ signal, and 3′ITR sequences come from a sub-group C adenovirus, more preferably from an adenovirus of serotype 2 (Ad2) or serotype 5 (Ad5).

On the other hand, in other embodiments, the expression viral vector can be carried out by using synthetic 5′ITR and/or 3′ITR; and also by using a 5′ITR and a 3′ITR which come from viruses of different serotype.

All other viral genes required for viral vector replication can be provided in trans within the virus-producing cells (packaging cells) as described below. Therefore, their inclusion in the nucleic acid construct of the expression viral vector is optional.

The expression viral expression vector herein described can be prepared and obtained by conventional methods known to those skilled in the art: Sambrook and Russell (Molecular Cloning: a Laboratory Manual; Third Edition; 2001 Cold Spring Harbor Laboratory Press); and Green and Sambrook (Molecular Cloning: a Laboratory Manual; Fourth Edition; 2012 Cold Spring Harbor Laboratory Press).

### Viral Particle

The disclosure also relates to a viral particle comprising an expression cassette as defined above or a vector as defined above. More precisely, the expression viral vector described above is packaged in the capsid formed by the capsid proteins, thereby constituting the viral particle of the disclosure.

As used herein, the term “viral particle” relates to an infectious and typically replication-defective virus particle comprising (i) the expression viral vector packaged within (ii) a capsid and, as the case may be, (iii) a lipidic envelope surrounding the capsid. The term “viral particle” can also encompasses the term “viral vector particle” or “recombinant vector particle”.

In preferred embodiments, the capsid is formed of capsid proteins of adeno-associated virus. In preferred embodiment, the viral vector particle is an AAV vector particle. As used herein, an AAV vector particle comprises at least 5′ITR and 3′ITR of an AAV genome and capsid proteins of adeno-associated virus. The term AAV vector particle encompasses any recombinant AAV vector particle or mutant AAV vector, genetically engineered.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

In a particular embodiment, a recombinant AAV vector particle of the invention may be prepared by encapsidating the expression viral vector of an AAV vector/genome derived from a particular AAV serotype on a viral particle formed by natural Cap proteins corresponding to an AAV of the same particular serotype. Nevertheless, several techniques have been developed to modify and improve the structural and functional properties of naturally occurring AAV viral particles (Bunning H et al. J Gene Med 2008; 10: 717-733).

Thus, in another AAV viral particle of the present disclosure, the viral expression vector including ITR(s) of a given AAV serotype can be packaged, for example, into: a) a viral particle constituted of capsid proteins derived from the same or different AAV serotype [e.g. AAV2 ITRs and AAV5 capsid proteins; AAV2 ITRs and AAV8 capsid proteins; AAV2 ITRs and Anc80 capsid proteins; AAV2 ITRs and AAV9 capsid proteins]; b) a mosaic viral particle constituted of a mixture of capsid proteins from different AAV serotypes or mutants [e.g. AAV2 ITRs with AAV1 and AAV5 capsid proteins]; c) a chimeric viral particle constituted of capsid proteins that have been truncated by domain swapping between different AAV serotypes or variants [e.g. AAV2 ITRs with AAV5 capsid proteins with AAV3 domains].

The skilled person will appreciate that the AAV viral particle according to the present disclosure may comprise capsid proteins from any AAV serotype. In a specific embodiment, the AAV viral particle comprises capsid proteins from a serotype selected from the group consisting of an AAV1, an AAV5, an AAV7, an AAV8, and an AAV9 which are more suitable for delivery to the liver cells (Nathwani et al. Blood 2007; 109: 1414-1421; Kitajima et al. Atherosclerosis 2006; 186:65-73).

In a particular embodiment, the AAV viral particle comprises capsid proteins from Anc80, a predicted ancestor of viral AAVs serotypes 1, 2, 8, and 9 that behaves as a highly potent gene therapy vector for targeting liver, muscle and retina (Zinn et al. Cell Reports 2015; 12:1-13). In a more particular embodiment, the viral particle comprises the Anc80L65 VP3 capsid protein (Genbank accession number: KT235804) or a capsid protein having at least 90% identity, 95% identity to the Anc80L65 VP3 capsid protein. Other VP capsid proteins of predicted ancestor of viral AAVs serotypes have been described in WO2015054653.

Virus-glycan interactions are critical determinants of host cell invasion. In a particular embodiment, the AAV viral particle comprises capsid proteins comprising one or more amino acids substitutions, wherein the substitutions introduce a new glycan binding site into the AAV capsid protein. In a more particular embodiment, the amino acid substitutions are in amino acid 266, amino acids 463-475 and amino acids 499-502 in AAV2 or the corresponding amino acid positions in AAV1, AAV3, AAV4, AAV5, AAV6, AAV7, AAV 8, AAV9, AAV10 or any other AAV serotype, also included Anc80 and Anc80L65. The introduced new glycan binding site can be a hexose binding site [e.g. a galactose (Gal), a mannose (Man), a glucose (Glu) or a fucose (fuc) binding site]; a sialic acid (Sia) binding site [e.g. a Sia residue such as is N-acetylneuraminic acid (NeuSAc) or N-Glycolylneuraminic acid (NeuSGc)]; or a disaccharide binding site, wherein the disaccharide is a sialic acid linked to galactose, for instance in the form of Sia(alpha2,3)Gal or Sia(alpha2,6)Gal. Detailed guidance to introduce a new binding site from an AAV serotype into a capsid protein of an AAV of another serotype is given on international patent publication WO2014144229 and in Shen et al. (J. Biol. Chem. 2013; 288(40):28814-28823). In a particular embodiment, the Gal binding site from AAV9 is introduced into the AAV2 VP3 backbone resulting in a dual glycan-binding AAV strain which is able to use both HS and Gal receptors for cell entry. Preferably, said dual glycan-binding AAV strain is AAV2G9. Shen et al. generated AAV2G9 by substituting amino acid residues directly involved and immediately flanking the Gal recognition site on the AAV9 VP3 capsid protein subunit onto corresponding residues on the AAV2 VP3 subunit coding region (AAV2 VP3 numbering Q464V, A467P, D469N, I470M, R471A, D472V, S474G, Y500F, and S501A).

In a particular embodiment, the viral particle comprises a viral expression vector wherein the 5′ITR and 3′ITR sequences are of an AAV2 serotype and the capsid proteins are of an Anc80 serotype.

In a more particular embodiment, the viral particle comprises a viral expression vector wherein:
the 5′ITR and 3′ITR sequences are of an AAV2 serotype,
the promoter is an alpha-1-antitrypsin promoter,
the terminator is a polyadenylation signal sequence, notably the bovine growth hormone polyadenylation signal,
the S/MAR sequences is of SEQ ID NO:2 or 12, and wherein the capsid proteins are of an Anc80 serotype.

In a more particular embodiment, the viral particle comprises a viral expression vector wherein:
the 5′ITR and 3′ITR sequences are of an AAV2 serotype,
the promoter is an alpha-1-antitrypsin promoter,
the terminator is a polyadenylation signal sequence, notably the bovine growth hormone polyadenylation signal,
the S/MAR sequences is of SEQ ID NO:2 or 12,
the transgene encodes argininosuccinate synthase 1,
   and wherein the capsid proteins are of an Anc80 serotype.

In a more particular embodiment, the viral particle comprises a viral expression vector wherein:
a 5′ITR sequence of SEQ ID NO:16 or 23,
an alpha-1-antitrypsin promoter of SEQ ID NO:19,
a MVM intron sequence of SEQ ID NO:21,
the transgene encoding argininosuccinate synthase 1 represented by SEQ ID NO:20,
S/MAR sequences of SEQ ID NO:2 or 12,
a polyadenylation signal sequence, notably the bovine growth hormone polyadenylation signal of SEQ ID NO:18,
and 3′ITR sequences of SEQ ID NO:17,
   and wherein the capsid proteins are of an Anc80 serotype.

In an alternative embodiment, the viral particle of the present disclosure may be an adenoviral particle, such as an Ad5 viral particle. As it is the case for AAV viral particle, capsid proteins of Ad viral particles can also be engineered to modify their tropism and cellular targeting properties, alternative adenoviral serotypes can also be employed.

### Pharmaceutical Composition

Another embodiment of the present disclosure is a pharmaceutical composition comprising an expression cassette as mentioned above, a vector as mentioned above, a host cell as mentioned above, or a viral particle as mentioned above, in combination with one or more pharmaceutical acceptable excipient, diluent or carrier, optionally comprising other active ingredients. Preferably the pharmaceutical composition comprises viral particles of the disclosure.

The amount of a composition (e.g. the amount of viral particles contained a pharmaceutical composition) that is administered to the subject or patient may vary depending on the particular circumstances of the individual subject/patient including, age, sex, and weight of the individual; the nature and stage of the disease, the aggressiveness of the disease; the route of administration; and/or concomitant medication that has been prescribed to the subject or patient. Dosage regimens may be adjusted to provide the optimum therapeutic response.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, a viral particle, a vector or a pharmaceutical composition can be administered to the patient for the treatment of a disease in an amount or dose comprised within a range between 1×10⁹ vg/kg and 5×10¹⁶ vg/kg, for example 1×10¹⁰ to 5×10¹⁴ vg/kg (vg: viral genomes; kg: subject's or patient's body weight). In a more particular embodiment, an amount comprised within a range of 1×10¹² to 1×10¹³ vg/kg is administered. In an alternative embodiment, an amount or dose comprised within a range of 1×10⁹ to 1×10¹¹ iu/kg (iu: infective units of the vector) is administered.

The pharmaceutical composition or medicament of the disclosure comprises a pharmaceutical carrier, diluent and/or adjuvant. Typically such composition or medicinal product comprises the viral particles in an effective amount, sufficient to provide a desired therapeutic effect, and a pharmaceutically acceptable carrier or excipient.

In a preferred embodiment, the disclosure relates then to a pharmaceutical composition that comprises viral particles as disclosed, and a pharmaceutically acceptable carrier. Any suitable pharmaceutically acceptable carrier or excipient can be used in the preparation of a pharmaceutical composition (See e.g. Remington: The Science and Practice of Pharmacy, Alfonso R. Gennaro (Editor) Mack Publishing Company, April 1997). Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Preferably, said pharmaceutical composition is formulated as a solution, more preferably as an optionally buffered saline solution.

In one embodiment, the pharmaceutical composition is a parenteral pharmaceutical composition, including a composition suitable for intravenous, intraarterial, subcutaneous, intraperitoneal or intramuscular administration. These pharmaceutical compositions are exemplary only and do not limit the pharmaceutical compositions suitable for other parenteral and non-parenteral administration routes.

In one embodiment, optionally in combination with one or more features of the various embodiments described above or below, the pharmaceutical composition carrying the viral particle is administered to the subject or patient by a parenteral route.

In one embodiment, the pharmaceutical composition is administered by intravenous, intraarterial, subcutaneous, intraperitoneal, intrahepatic or intramuscular route, preferably intrahepatic route.

In one embodiment, the pharmaceutical composition is administered by interstitial route, i.e. by injection to or into the interstices of a tissue.

### Use

Cassette, vector, host cell, viral particle and pharmaceutical composition of the disclosure have in vivo therapeutic utilities. For example, they can be administered to cells in culture, e.g. in vitro or in vivo, or in a subject, e.g. in vivo, to treat, or prevent a variety of disorders. Cassette, vector, host cell, viral particle and pharmaceutical composition of the disclosure allow notably the maintenance of the viral genomes after administration in a subject in need thereof. More specifically, when viral particle or pharmaceutical composition is administered by intrahepatic route (e.g. in the liver) in neonate, infant or child, viral vectors multiply at the same time as the hepatocytes do when the neonates or infantile liver grows.

It is the contemplated herein the use of the cassette, vector, host cell, viral particle and pharmaceutical composition of the disclosure, as a medicament. Preferably they are used in gene therapy in subjects in need thereof. More preferably they are used for the treatment of diseases with hepatic origin.

As used herein, “treat” or “treating” or “treatment” means to administer the medicament, such as the pharmaceutical composition containing the viral particles as disclosed herein, to a patient having one or more disease symptoms for which the medicament has a known therapeutic activity. Typically, the pharmaceutical composition is administered in an amount effective to alleviate one or more disease symptoms in the treated patient, whether by inducing the regression of or inhibiting the progression of such symptom(s) by any clinically measurable degree.

As used herein, the term “diseases with hepatic origin” refers to genetic diseases caused by abnormal expression of a gene in the liver. Such diseases with hepatic origin include without limitation: Wilson disease, Phenylketonuria, Propionic 19cademia, Hemophilia A and B, Progressive familiar intrahepatic cholestasis 1, -2 and 3, urea cycle disorders such as Citrullinemia type 1, Argininosuccinic aciduria, Arginase deficiency, Ornithine translocase deficiency, Citrin deficiency, Ornithine transcarbamylase deficiency, N-acetylglutamte synthase deficiency, Galactosemia type 1, 2, 3, Tyrosinemia type 1, 2, 3, Alagille syndrome, Gaucher disease, Glycogen storage diseases such as Pompe disease, Crigler Najar, Hemochromatosis, alpha 1-antitrypsin deficiency, cystic fibrosis, Hypercholesterolemia, Mucopolysaccharidose (MPS-1), Hunter syndrome (MPS-2), Sanfilippo syndrome (MPS-3), Fabry disease, Homocystinuria, Non-alcoholic steatohepatitis Wolman disease, Niemann Pick type A-C, Mitochondrial DNA depletion syndromes, and Primary hyperoxaluria. Preferably such disease is citrullinemia, preferably citrullinemia type 1.

As used herein, the term “subject” refers to a mammal and in particular a human. This also encompasses the term “patient”. Said subject can be a neonate, an infant, a child or an adult, preferably a neonate, an infant or a child, more preferably a neonate or an infant. As used herein “neonate” refers to a person having until 28 days. As used herein, the term “infant” refers to a person having between 29 days until 2 years.

In a preferred embodiment, the present disclosure relates to the above-mentioned cassette, vector, host cell, viral particle and pharmaceutical composition of the disclosure for use in the treatment of citrullinemia, notably citrullinemia type 1, in neonate or infant.

In another embodiment, the present disclosure relates to a method for the treatment of diseases with hepatic origin, notably citrullinemia, preferably citrullinemia type 1, comprising the administration to a patient of a therapeutically effective amount of an expression cassette as defined above, or a vector as defined above, a host cell as defined above, or a viral particle as defined above, or a pharmaceutical composition as defined above.

In another embodiment, the present disclosure also relates to the use of an expression cassette as defined above, or a vector as defined above, a host cell as defined above, or a viral particle as defined above, or a pharmaceutical composition as defined above, in the preparation of a medicament for the treatment of diseases with hepatic origin, notably citrullinemia, preferably citrullinemia type 1.

According to the disclosure, a cassette as defined above, or a vector as defined above, a host cell as defined above, or a viral particle as defined above, or a pharmaceutical composition as defined above, may be administered as the sole active ingredients or in conjunction with, e.g. as an adjuvant to or in combination to other drugs e.g. anti-inflammatory agents for the treatment or prevention of diseases mentioned above, compounds that enhance vector stability, agents that are used to prevent disease progression or symptoms treatment.

In another embodiment, a cassette as defined above, or a vector as defined above, may also be administered in conjunction with a demethylating agent. Said cassette or vector and said demethylating agent can be administered simultaneously, separately or spread over time for the treatment or prevention of diseases mentioned above.

Another embodiment is also a kit comprising an expression cassette as defined above, or a vector as defined above, a host cell as defined above, or a viral particle as defined above, or a pharmaceutical composition as defined above, in one or more containers, optionally further comprising instructions or packaging materials. The kit may include instructions or packaging materials that describe how to administer the viral vector particle contained within the kit to a patient. Containers of the kit can be of any suitable material, e.g. glass, plastic, metal, etc., and of any suitable size, shape, or configuration. In certain embodiments, the kits may include one or more ampoules or syringes that contain the viral particle or pharmaceutical composition comprising such viral particle in a suitable liquid or solution form.

### Production of Viral Particles

Production of viral particles carrying the expression viral vector as disclosed above can be performed by means of conventional methods and protocols, which are selected taking into account the structural features chosen for the actual embodiment of the expression cassette, nucleic acid construct of the vector and viral particle of the vector to be produced.

Briefly, viral particles can be produced in a specific virus-producing cell (packaging cell), which is transfected with the nucleic acid construct of the vector to be packaged, in the presence of a helper vector or virus or other DNA construct(s).

Typically, a process of producing viral particles comprises the following steps:

a) culturing a packaging cell comprising an viral expression vector as described above in a culture medium under suitable conditions for producing; and

b) harvesting the viral particles from the cell culture supernatant and/or inside the cells.

Said packaging cells (also referred as “host cells”) can be adherent or suspension cells. The packaging cell, and helper vector or DNA constructs provide together in trans all the missing functions which are required for the complete replication and packaging of the viral vector. For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells. As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124). In another embodiment, the packaging cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, optionally in combination with one or more features of the various embodiments described above or below, the host cell comprises:
An expression viral vector (e.g. the recombinant AAV genome), generally as a plasmid;
a nucleic acid construct, for example a plasmid, encoding AAV rep and/or cap genes which does not carry the ITR sequences; and/or
a nucleic acid construct, for example a plasmid or virus, comprising viral helper genes.

Viral genes necessary for AAV replication are referred herein as viral helper genes. Typically, said genes necessary for AAV replication are adenoviral helper genes, such as E1A, E1B, E2a, E4, or VA RNAs. Preferably, the adenoviral helper genes are of the Ad5 or Ad2 serotype.

Conventional methods can be used to produce viral particles of the AAV vector, which consist on transient cell co-transfection with nucleic acid construct (e.g. a plasmid) carrying the recombinant AAV vector/genome of the invention; a nucleic acid construct (e.g. an AAV helper plasmid) that encodes rep and cap genes, but does not carry ITR sequences; and with a third nucleic acid construct (e.g. a plasmid) providing the adenoviral functions necessary for AAV replication.

Thus, in a particular embodiment, said host cell is characterized by comprising:
an expression viral vector as disclosed above (e.g., a recombinant AAV expression vector);
a nucleic acid construct encoding AAV rep and cap genes which does not carry the ITR sequences; and
a nucleic acid construct comprising adenoviral helper genes.

Alternatively, the rep, cap, and adenoviral helper genes can be combined on a single plasmid (Blouin V et al. J Gene Med. 2004; 6(suppl): S223-S228; Grimm D. et al. Hum. Gene Ther. 2003; 7:839-850). Thus, in another particular embodiment, said host cell is characterized by comprising:
an expression viral vector as disclosed above (e.g. an AAV expression viral vector or an expression cassette as defined above); and
a nucleic acid construct encoding AAV rep and cap genes, which does not carry the ITR sequences and further comprising adenoviral helper genes.

In a further particular embodiment, optionally in combination with one or more features of the various embodiments described above or below, the host cell comprises:
an expression viral vector as disclosed above (e.g. a recombinant AAV expression viral vector);
a nucleic acid encoding AAV rep and cap genes, which does not carry the ITR sequences; and
a nucleic acid comprising adenoviral helper genes E2a, E4, and VA RNAs,
   wherein co-transfection is performed in cells, preferably mammalian cells, that constitutively express and transcomplement the adenoviral E1 gene, like HEK-293 cells (ATCC CRL-1573).

Large-scale production of AAV vectors according to the disclosure can also be carried out for example by infection of insect cells with a combination of recombinant baculoviruses (Urabe et al. Hum. Gene Ther. 2002; 13: 1935-1943). SF9 cells are co-infected with three baculovirus vectors respectively expressing AAV rep, AAV cap and the AAV vector to be packaged. The recombinant baculovirus vectors will provide the viral helper gene functions required for virus replication and/or packaging. By using helper plasmids encoding the rep ORF (open reading frame) of an AAV serotype and cap ORF of a different serotype AAV, it is feasible packaging a vector flanked by ITRs of a given AAV serotype into virions assembled from structural capsid proteins of a different serotype. It is also possible by this same procedure to package mosaic, chimeric or targeted vectors.

Smith et al 2009 (Molecular Therapy, vol. 17, no. 11, pp 1888-1896) further describes a dual baculovirus expression system for large-scale production of AAV vectors in insect cells. On the other hand, the production of HC-Ad vectors according to the invention can be carried out by means of mammalian cells that constitutively express and transcomplement the adenoviral E1 gene, and also Cre recombinase (e.g. 293Cre cells). These cells are transfected with the HC-Ad vector genome and infected with a first-generation adenoviral helper virus (E1-deleted) in which the packaging signal is flanked by loxP sequences. [Parks R J et al. Proc. Natl. Acad. Sci. USA 1996; 13565-13570; for 293Cre cells, see Palmer and Engel. Mol. Ther. 2003; 8:846-852]. Several Cre/loxP-based helper virus systems have been described that can be used for packaging HC-Ad vectors, such as AdAdLC8cluc, or the optimized self-inactivating AdTetCre helper virus (EP2295591; Gonzalez-Aparicio et al. Gene Therapy 2011; 18: 1025-1033).

Further guidance for the construction and production of viral vectors according to the disclosure can be found in:
Viral Vectors for Gene Therapy, Methods and Protocols. Series: Methods in Molecular Biology, Vol. 737. Merten and Al-Rubeai (Eds.); 2011 Humana Press (Springer).
Gene Therapy. M. Giacca. 2010 Springer-Verlag.
Heilbronn R. and Weger S. Viral Vectors for Gene Transfer: Current Status of Gene Therapeutics. In: Drug Delivery, Handbook of Experimental Pharmacology 197; M. Schäfer-Korting (Ed.). 2010 Springer-Verlag; pp. 143-170.
Adeno-Associated Virus: Methods and Protocols. R. O. Snyder and P. Moulllier (Eds). 2011 Humana Press (Springer).
Bünning H. et al. Recent developments in adeno-associated virus technology. J. Gene Med. 2008; 10:717-733.
Adenovirus: Methods and Protocols. M. Chinon and A. Bosch (Eds.); Third Edition. 2014 Humana Press (Springer).

Moreover, the construction of recombinant AAV viral particles is generally known in the art and has been described for instance in U.S. Pat. Nos. 5,173,414 and 5,139,941; WO 92/01070, WO 93/03769, (Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. And Immunol. 158:97-129; and Kotin, R. M. (1994) Human Gene Therapy 5:793-801.

Suitable culture media will be known to a person skilled in the art. The ingredients that compose such media may vary depending on the type of cell to be cultured. In addition to nutrient composition, osmolarity and Ph are considered important parameters of culture media. The cell growth medium comprises a number of ingredients well known by the person skilled in the art, including amino acids, vitamins, organic and inorganic salts, sources of carbohydrate, lipids, trace elements (CuS04, FeS04, Fe(N03)3, ZnS04 . . . ), each ingredient being present in an amount which supports the cultivation of a cell in vitro (i.e., survival and growth of cells). Ingredients may also include different auxiliary substances, such as buffer substances (like sodium bicarbonate, Hepes, Tris . . . ), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

### Sequences of the Disclosure

Cloned Sequences of the disclosure are described in Table 1 below. Reverse sequences of IFN-β, DHFR, Apo-B, CPSi, c-Myc and KSHV are the respective reverse complementary of the forward sequences.

TABLE 1
Sequences of the disclosure

| SEQ ID | |
|---|---|
| NO: | Describes the amino acid or nucleotide sequence below: |
| SEQ ID | 5′ |
| NO: 1 | GGTGCCACCCCCTCCACCTTGGACACAGGACGCTGTGGTTTCTGAGCCAGGTACAATGACTCCTTTCGGT |
| (eGFP) | AAGTGCAGTGGAAGCTGTACACTGCCCAGGCAAAGCGTCCGGGCAGCGTAGGCGGGACTCAGATCCCAGC |
| | CAGTGGACTTAGCCCCTGTTTGCTCCTCCGATAACTGGGGTGACCTTGGTTAATATTCACCAGCAGCCTC |
| | CCCCGTTGCCCCTCTGGATCCACTGCTTAAATACGGACGAGGACAGGGCCCTGTCTCCTCAGCTTCAGGC |
| | ACCACCACTGACCTGGGACAGTGAA-3′ |
| SEQ ID | 5′- |
| NO: 2 | AGATCTAAATAAACTTATAAATTGTGAGAGAAATTAATGAATGTCTAAGTTAATGCAGAAACGGAGAGAC |
| (IFN-β- | ATACTATATTCATGAACTAAAAGACTTAATATTGTGAAGGTATACTTTCTTTCCACATAAATTTGTAGTC |
| Forward) | AATATGTTCACCCCAAAAAAGCTGTTTGTTAACTTGCCAACCTCATTCTAAAATGTATATAGAAGCCCAA |
| | AAGACAATAACAAAAATATTCTTGTAGAACAAAATGGGAAAGAATGTTCCACTAAATATCAAGATTTAGA |
| | GCAAAGCATGAGATGTGTGGGGATAGACAGTGAGGCTGATAAAATAGAGTAGAGCTCAGAAACAGACCCA |
| | TTGATATATGTAAGTGACCTATGAAAAAAATATGGCATTTTACAATGGGAAAATGATGATCTTTTTCTTT |
| | TTTAGAAAAACAGGGAAATATATTTATATGTAAAAAATAAAAGGGAACCCATATGTCATACCATACACAC |
| | AAAAAAATTCCAGTGAATTATAAGTCTAAATGGAGAAGGCAAAACTTTAAATCTTTTAGAAAATAATATA |
| | GAAGCATGCCATCATGACTTCAGTGTAGAGAAAAATTTCTTATGACTCAAAGTCCTAACCACAAAGAAAA |
| | GATTGTTAATTAGATTGCATGAATATTAAGACTTATTTTTAAAATTAAAAAACCATTAAGAAAAGTCAGG |
| | CCATAGAATGACAGAAAATATTTGCAACACCCCAGTAAAGAGAATTGTAATATGCAGATTATAAAAAGAA |
| | GTCTTACAAATCAGTAAAAAATAAAACTAGACAAAAATTTGAACAGATGAAAGAGAAACTCTAAATAATC |
| | ATTACACATGAGAAACTCAATCTCAGAAATCAGAGAACTATCATTGCATATACACTAAATTAGAGAAATA |
| | TTAAAAGGCTAAGTAACATCTGTGGCAATATTGATGGTATATAACCTTGATATGATGTGATGAGAACAGT |
| | ACTTTACCCCATGGGCTTCCTCCCCAAACCCTTACCCCAGTATAAATCATGACAAATATACTTTAAAAAC |
| | CATTACCCTATATCTAACCAGTACTCCTCAAAACTGTCAAGGTCATCAAAAATAAGAAAAGTCTGAGGAA |
| | CTGTCAAAACTAAGAGGAACCCAAGGAGACATGAGAATTATATGTAATGTGGCATTCTGAATGAGATCCC |
| | AGAACAGAAAAAGAACAGTAGCTAAAAAACTAATGAAATATAAATAAAGTTTGAACTTTAGTTTTTTTTA |
| | AAAAAGAGTAGCATTAACACGGCAAAGCCATTTTCATATTTTTCTTGAACATTAAGTACAAGTCTATAAT |
| | TAAAAATTTTTTAAATGTAGTCTGGAACATTGCCAGAAACAGAAGTACAACAGCTATCTGTGCTGTCGCC |
| | TAACTATCCATAGCTGATTGGTCTAAAATGAGATACATCAACGCTCCTCCATGTTTTTTGTTTTCTTTTT |
| | AAATGAAAAACTTTATTTTTTAAGAGGAGTTTCAGGTTCATAGCAAAATTGAGAGGAAGGTACATTCAAG |
| | CTGAGGAAGTTTTCCTCTATTCCTAGTTTACTGAGAGATTGCATCATGAATGGGTGTTAAATTTTGTCAA |
| | ATGCTTTTTCTGTGTCTATCAATATGACCATGTGATTTTCTTCTTTAACCTGTTGATGGGACAAATTACG |
| | TTAATTGATTTTCAAACGTTGAACCACCCTTACATATCTGGAATAAATTCTACTTGGTTGTGGTGTATAT |
| | TTTTTGATACATTCTTGGATTCTTTTTGCTAATATTTTGTTGAAAATGTTTGTATCTTTGTTCATGAGAG |
| | ATATTGGTCTGTTGTTTTCTTTTCTTGTAATGTCATTTTCTAGTTCCGGTATTAAGGTAATGCTGGCCTA |
| | GTTGAATGATTTAGGAAGTATTCCCTCTGCTTCTGTCTTCTGAAAGAGATTGTAGAAAGTTGATACAATT |
| | TTTTTTTCTTTAAATATTTGATAGAATTC-3′ |
| SEQ ID | 5′- |
| NO: 3 | TGTGAAGAGACACCATGACCACAGAAACTCTTATAAAGGAAAGCAATTATTGGGTCCAGCTTACAGTTCA |
| (DHFR- | GAGGTTTAATCCATTGTCATGATTGCAGGAAGTATGGTGCGCCACAGGCAGACATGGTGCTGGAGAAGTA |
| Forward) | GATGAGAGTTCTATATCAGATTGACACACTTCTTCCAACAAGGCCACACCTCCACTCACTCTGAGCCTAT |
| | GGGGCCATTTTCATTCAAACCACCAAAGCTACAAGGTAGCTTATACCCCAGCTTGCTATTTCTGATGAGA |
| | CTTAGTAAATAGTCTTAAAAGCCCATAAAATGACTCAAAACTAGTTTTTTTATTATTATTATTAGTTCAA |
| | ATTAGGAAGAAGCTTGCTTTACATGTCAATCCCTTCTCCCTCTCCCTCATCAAAACTAGTTTTTTGTTTT |
| | TTAGGTTTTTTTTCAAGACAGGGTTTCTCTGTGTAGCTTTGGAGCCTATCCTGGCACTCGCTCTGGAGAC |
| | CAGGCTGGCCTCGAACTCACAGAGATCTGCCTGCCTTTGCCTCCCGAGTCCTGGGATTAAAGGCATGCAC |
| | CACCAACACCTGGCCAAAATTAGTTTTAAGTTCCAGTTCTAGGAGCCTCCAATGCCCTCTTTTGGCTTCC |
| | ATGGGAACCAGGAACACTATATATATATATATATATATATATATATATATATATATATATATATTCAGGC |
| | AAATATTTATGCATATAAAAATAAAATAAATCTTTTTTCCTTTTTTTTTTAAAGAAGTGCAATTGTCTTG |
| | GAATTTTTGTGGCTGCTCTGCCCTTATGTGTAACTGGACACTACCAGCATCTAAACACTGGCCTGAAACC |
| | AGCCAAAGAAAACCTTTGTGCCAGGTCCTGTGTCAAAGTATTATGTTCCTTTTAGGATATCCTATATCGT |
| | AAAGGATTTATTTTACTGATAGCATCTTAACTTCCTTTGAAAGGTTGGTCTTCTCAAGCAGTCCTCGTGG |
| | AGCTGGCTCCTCAGCTAATGCCAGGGGACAATAATGATCCCCTCCCAAAACCAAACAGAAAACCATGGCA |
| | ACTCTGGTTTCCTTGGGCAGCACCTGCTTTAAGAATGAGCAAATGACCAATCAGCTCATGAAACTAAATA |
| | CTCTATTATTACTAAAATATTTTTTTGAGACAGGGCATGGAATTCATCACATAGTTCAGGTTGGCCTTGA |
| | ACTCAGAGAGACTCACTTACCTTTGCCTCCCACGTGCTGGAATTAAAGGCATGAACCACCACACCAAACA |
| | TAACACTTGAATTTTGGAAGAGTCCTTCTTCCAATAGATTTGAGGTTTTGAAAATGTGGCACAGAAAATA |
| | TGAATTCAAATATAATGAAAACAAGAGATAACTTTCAACTAAGTTTCTATAGGTTCTTGCTAGGAATCCT |
| | AAGCTTGTCTGAAACTCTAGAGCTTCTGTTTCTAGTCTTCTGAGTGTTAGTATTGTAGGTATGTGCCCTG |
| | CCTCAGTGTGATGTTTTTGATAATCTTAAAGAAATCAAAGAAATTTTATAAAAGACTAGACTGTGCTACA |
| | CAAAAAGAATATTCAGATGCCAAGAAAGAGTTCTTAGAAATTAAGAAATATGCTACTAGTATAAATCCTT |
| | TATAAAGTGGAATGACAAATCTGATGAAATCTTACTAAAAGTAGAAAAACATAAACATCAAAGACATGAA |
| | TAATAAGAAAATCATATTGTGCATATCATTAACCTAAAACATTAACTTGCAAAAATAGAATAGTCCAAAA |
| | AGTAAACAAAATAAATAAATCACCAAGACATGATACAAGGACAATTCCTAGAATGATAAAACAAGAATAT |
| | TCATTATAAAAGGCCCTATCACTAAAGCACAACAGAAACAGACTCAAAAGATAAATCTTCATTGTCACTG |
| | GAGAGAAGTCATACTATCATAGCACTCAGAAGGAAATAAAAATCAAAATGTCAAAAAGGACCTCAGCCTC |
| | TGAAACACAAATACAAAATATGTCCGCCTTCTTGACAGGCATTACTCTTCAATTAACATTTTAAGAAAAC |
| | TATAAAGAGAGCTTAGTATTTTAAGAAATCTGTAGCTATTTCTTTTATAAGCATGACAACTAAGTTTCCT |
| | GATTTAAACAGACCTAAAAAACCGGTGAAGTGAGTGGAGAAAGGGGATACGAAGACAGCATCCCACATGA |
| | CTGCTCCCAGTAAAGGCAAGGTCTTCATCCATTTTATCCTGAACTCTGGGAAATTTATAAAGAACAGAAA |
| | TGTATTTCTCTCAGTTCTGGAGCCTCAGTCCAGGACACTAAGTCTAGGTACTACACTCTCACATGGTGGA |
| | AACTAGAAAGCAAGCTCACTTGTCACTCACTACCTGATGCCTCTT-3′ |
| SEQ ID | 5′- |
| NO: 4 | GAATTCAAAAAGATGAGGTAATTGTGTTTTTATAATTAAATATTTTATAATTAAAATATTTATAATTAAA |
| (Apo-B- | ATATTTATAATTAAATATTTTATAATTAAAATATTTATAATTAAATATTTTATAATTAAAATATTTATAA |
| Forward) | TTAAATATTTTATAATTAAAATGTTTATAATTAAATATTTTATAATTAAAATGTTTATAATTACATATTT |
| | TATAATTAAAATGTTTATAATTACATATTTTATAATTAAAATGTTTATAATTACATATTTTATAATTACA |
| | TATTTTATAAAGTATTTATAATTACATATTTTATAATTAAAGTATTTATAATTACATATTTTATAATTAA |
| | AGTATTTATAATTACATATTTTATAATTCAATATTTTATAAATAGTTAAAAAGACGAGGAAAAAATTAAA |
| | AAGACGAGGTTATTGATCTCAGGAATTGTATTTGCCAAGTGAGAAGGAAAAAATATTCACAAAGGCTTGT |
| | AGAATTC-3′ |
| SEQ ID | 5′- |
| NO: 5 | GAATTCGGATCCCATTCTCCTTGATGTACTAATTTTTCTTTAAAAGTGATAATAATAGCTCCCATTTAGA |
| (CPSi- | ATTTTTAAATAACACAACAAATGTAAAGTAACTAATGTGTCCTCTGGATCATGGTAAGTAATGAATAAAT |
| Forward) | TTAACTCCCTTTACCTTCTCCCTTTGCTATTTTTTCCATGCTAGGATTTATACATTTTTAAAAAACTAAA |
| | TCTGCTATCAAATGACAGCTTTAAATTTACTTTTTAAAATTTGTTATTGTATATATTTATGGGGTATAAA |
| | GTGATGTTATGATATATATATACACAATGTACACTGATTAAATCAAGCCAATTAACATTTTATCATCTCA |
| | AATACTTAACATTTTTTGTAGTGAGAACATTTGAAATTTACTTTTAGCAATTTCAAAACATACATTATTA |
| | TTATTAACTATAGTCACCATGATGTACCATAGATCTTTAAAAACTTATTCTTCCTGCCTAACTGAAACTT |
| | TGTACTCTTTGACTAACATCTTTTCATTCCCCCACTTCCCAGCCTCTGGTAATCACCATTACACACTCTG |
| | CTTCTATGAGTTCAATTGCTTTAGACTCCACGTAATAAATGAGATCATGCAGCATTTGGCTTTCTGTGCT |
| | GGCTTATCCTTGCTTAGCATGGTGTCTTACAGGTTCATCCATGTTGCAACAAATAACAGAATCTCATTCT |
| | TTGTTAAGGCTGAATACTATTCCATTGGGTATATATACCACATTTTCCTTATCCATTAATCCACTGATGG |
| | ACCCTTAGGTTGTTGATTCCATATATTGGCTATTGTAAATAGTGCAGCAATGAACATGAGAGTGCAACTA |
| | TCTCTTCAATGTACTGATTTCGAATCCTTCGGATCTATCTCAGAAGTGAGATTGCAGGATCATATAATTC |
| | TACTTTTAGTCTTTTGAGGAGCTCCATACAGCTTTCCATATGGCCATACTAATTACATTCTCATCAACAG |
| | TGTACAATGGTTTCCTTTTCTCCACATCCTCACCAACATTTATAATTTTTTGTCTTTTTGATAATAGCCA |
| | TCTGACAGGTGTAAAGTGATAGCTCATTGCAGTTTTAATTTGCATTTTTTGATGATTAGTAATGTTGAGA |
| | ATTTTTTCATATATCTCTTGGCCAGTTGCATGTCTTCTTTGGAAAAATGTCTATTCAGTTCCTTTGCCCA |
| | TTTTTTAATTGGGATTTTTGGTTTCTTGCTATTGAGTTGTTTGAATTC-3′ |
| SEQ ID | 5′- |
| NO: 6 | GAATTCCGATGTTGTTTCTGTGGAAAAGAGGCAGGCTCCTGGCAAAAGGTCAGAGTCTGGATCACCTTCT |
| (C-Myc- | GCTGGAGGCCACAGCAAACCTCCTCACAGCCCACTGGTCCTCAAGAGGTGCCACGTCTCCACACATCAGC |
| Forward) | ACAACTACGCAGCGCCTCCCTCCACTCGGAAGGACTATCCTGCTGCCAAGAGGGTCAAGTTGGACAGTGT |
| | CAGAGTCCTGAGACAGATCAGCAACAACCGAAAATGCACCAGCCCCAGGTCCTCGGACACCGAGGAGAAT |
| | GTCAAGAGGCGAACACACAACGTCTTGGAGCGCCAGAGGAGGAACGAGCTAAAACGGAGCTTTTTTGCCC |
| | TGCGTGACCAGATCCCGGAGTTGGAAAACAATGAAAAGGCCCCCAAGGTAGTTATCCTTAAAAAAGCCAC |
| | AGCATACATCCTGTCCGTCCAAGCAGAGGAGCAAAAGCTCATTTCTGAAGAGGACTTGTTGCGGAAACGA |
| | CGAGAACAGTTGAAACACAAACTTGAACAGCTACGGAACTCTTGTGCGTAAGGAAAAGTAAGGAAAACGA |
| | TTCCTTCTAACAGAAATGTCCTGAGCAATCACCTATGAACTTGTTTCAAATGCATGATCAAATGCAACCT |
| | CACAACCTTGGCTGAGTCTTGAGACTGAAAGATTTAGCCATAATGTAAACTGCCTCAAATTGGACTTTGG |
| | GCATAAAAGAACTTTTTTATGCTTACCATCTTTTTTTTTTCTTTAACAGATTTGTATTTAAGAATTGTTT |
| | TTAAAAAATTTTAAGATTTACACAATGTTTCTCTGTAAATATTGCCATTAAATGTAAATAACTTTAATAA |
| | AACGTTTATAGCAGTTACACAGAATTTCAATCCTAGTATATAGTACCTAGTATTATAGGTACTATAAACC |
| | CTAATTTTTTTTATTTAAGTACATTTTGCTTTTTAAAGTTGATTTTTTTCTATTGTTTTTAGAAAAAATA |
| | AAATAACTGGCAAATATATCATTGAGCCAAATCTTAAGTTGTGAATGTTTTGTTTCGTTTCTTCCCCCTC |
| | CCAACCACCACCATCCCTGTTTGTTTTCATCAATTGCCCCTTCAGAGGGCGGTCTTAAGAAAGGCAAGAG |
| | TTTTCCTCTGTTGAAATGGGTCTGGGGGCCTTAAGGTCTTTAAGTTCTTGGAGGTTCTAAGATGCTTCCT |
| | GGAGACTATGATAACAGCCAGAGTTGACAGTTAGAAGGAATGGCAGAAGGCAGGTGAGAAGGTGAGAGGT |
| | AGGCAAAGGAGATACAAGAGGTCAAAGGTAGCAGTTAAGTACACAAAGAGGCATAAGGACTGGGGAGTTG |
| | GGAGGAAGGTGAGGAAGAAACTCCTGTTACTTTAGTTAACCAGTGCCAGTCCCCTGCTCACTCCAAACCC |
| | AGGAATTC-3′ |
| SEQ ID | 5′- |
| NO: 7 | GAATTCGGAGGGGGATCCCGGCGCGCCACCCTCCCCGGCAACAACCTGTTGCCATGTATGGCGATTTGTA |
| (KSHV- | TCAGTCACAAGCACACAACCCCTGCTAGTATTAATGGTGTTTAAAACGTTCTACACGTACGGCGGACCGC |
| Forward) | ATCCGTCGCAAGCACGCGCATATAACCCCCAAATGCACCATGATGAGAAGCACAGCCACGCGTCAAAAAA |
| | CTTTAAAAACATCGTTATCCAATATCATTAAAAACCACACCGAAATTTACACAGGTAGCACGTCACCGTG |
| | TTAGTGTCACCCACTGTACACAAGGCGTGTCGTATATGTAGTATAGGTATTTGATGAGGCGGAAGCATAT |
| | CCCGCTTCCAGCGAACGGAAATAAGAATCATCCGTTCCAGCATTTATTCAAAGAGGGCACAGAGGATTCA |
| | CATTGTTTAGAGAGAGTTTTTCTTAGTCACCATTCCATACTTGGGCAGTATTGGCCTACGATTTGGGCGA |
| | CGTTTCAGGCTGGTCTATTCTCCGTCCACTTTTCCCCGGCTATTCTGTCCCAGCATAGGCTCTTGAAATA |
| | AACAATGTTTACCGAGTAAAAGGTTCCACTCACCCTCATTTGTCGTTGCACCCATCCCCCCTTTGCTTAA |
| | TCACCCGAAAACTAGAGGACACGGATGGAAAACATATCGCACGCGGGTTGTTTGAAAGTCAACAGCTACT |
| | TGTTTTTAATGAGGACAGATTTGGGCACAGGCCAGAGGGTAAAGCCCTACGTGTGCGCGGGGGGGGGGGT |
| | GTATACGCTGCGAAAACCTGCACGGTGCATAACACCCAGGGCGTCACGTCACATATCTCTGTGCACCCAA |
| | GTGGTTGTTCAACCGTTGTTTTTTGGATGATTTTTCCGCACCGGCTTTTTTGTGGGCGCGCATAGGTCGG |
| | TACGCGCTGTCCCCCTAAGTCCCGCACGGTCGTTCGGGCCCCCGTCCGGCTCGTCTCCGGATGAACCGTC |
| | ACGTTCTTTGTCTCCAGAGGCGACGTCTCCTTCAGATGACTCGTCCGTGGGCTCCTCGTCCGTCCCGCCC |
| | GCGGGTCCGACAAGGACCGTCAATTCGATGTTATCTTCGTTCGCGGTTGGCCGGCGCGGCCGTCGGTATG |
| | GCAGTACGGTCACCCGGGTGTTATTTGCCGCGTATAATGCCCTCACAGTGCCACTTACGCGGCATATGCC |
| | GCCAAATGCAAACACAATAAATATTTGGTAAAACCCAAAGAAGCAGAGAAAACCGAGCACGGCCCCGGGG |
| | GAGAATGTTCCCGCAGGAGCAGTTAGGATGACCAGGAGCGTCCAGGTGCACAACGCCACGCCGACAAGCC |
| | CAGCCACCACCACAGACATCAGCAGAAACAGTTCAAAAATTTCTTGGCGCTCCATCTCCGGCCACAGGTT |
| | AAGGCGACTACGCCACTGCGTGCGCGTGCGGTATATAACGCGACACATTTGACAGGCCGTGTTTCGAGAC |
| | ACTGTTAGCCAAGTGCTTAAACACTGCGGGTGGACGACATCCAGCTCTCCGGTACAGGCGCAGGGGTGTA |
| | TGCCCTCGTTCCCCACCTCTTCCCTACATATCCAGCAGATGGGTCCCTCTACACCCTCTTCTACGTCCTT |
| | AGACGCCATCTCTGCAGCTGGGGTGGAAGTCTGAAAAAGGGAAAGGGGAGGTGAGCAGAGTGCCCAGTTA |
| | GTCTCCGACCCGCCGTCCGCCCTACTGTCGCTATCCCGCCTTGACAGATGTCTAACGTATTCACGGACGC |
| | CACATGTGTGTCTATTTTCCTACATCCAGGCTTTCCCTGGAAAACTGTCACAACCCACCCTGCTTTAGCT |
| | CTACATCTGTATTTTTGTTTACGCACAGGATCAACGCTTCGTGCCCGTCCACCCCCGCGCTCTCCGCCTG |
| | TGTTTGGAGGTTTTATGAGTGGTTAGTTCTAGGCAGCTCCGGACAAGTTGTCCAAAACACGGCGCGCCCC |
| | GCCCTTCCTTCCCTCCGAATTC-3′ |
| SEQ ID | GAATTCTATCAAATATTTAAAGAAAAAAAAATTGTATCAACTTTCTACAATCTCTTTCAGAAGACAGAAG |
| NO: 8 | CAGAGGGAATACTTCCTAAATCATTCAACTAGGCCAGCATTACCTTAATACCGGAACTAGAAAATGACAT |
| (IFN-β- | TACAAGAAAAGAAAACAACAGACCAATATCTCTCATGAACAAAGATACAAACATTTTCAACAAAATATTA |
| Reverse) | GCAAAAAGAATCCAAGAATGTATCAAAAAATATACACCACAACCAAGTAGAATTTATTCCAGATATGTAA |
| | GGGTGGTTCAACGTTTGAAAATCAATTAACGTAATTTGTCCCATCAACAGGTTAAAGAAGAAAATCACAT |
| | GGTCATATTGATAGACACAGAAAAAGCATTTGACAAAATTTAACACCCATTCATGATGCAATCTCTCAGT |
| | AAACTAGGAATAGAGGAAAACTTCCTCAGCTTGAATGTACCTTCCTCTCAATTTTGCTATGAACCTGAAA |
| | CTCCTCTTAAAAAATAAAGTTTTTCATTTAAAAAGAAAACAAAAAACATGGAGGAGCGTTGATGTATCTC |
| | ATTTTAGACCAATCAGCTATGGATAGTTAGGCGACAGCACAGATAGCTGTTGTACTTCTGTTTCTGGCAA |
| | TGTTCCAGACTACATTTAAAAAATTTTTAATTATAGACTTGTACTTAATGTTCAAGAAAAATATGAAAAT |
| | GGCTTTGCCGTGTTAATGCTACTCTTTTTTAAAAAAAACTAAAGTTCAAACTTTATTTATATTTCATTAG |
| | TTTTTTAGCTACTGTTCTTTTTCTGTTCTGGGATCTCATTCAGAATGCCACATTACATATAATTCTCATG |
| | TCTCCTTGGGTTCCTCTTAGTTTTGACAGTTCCTCAGACTTTTCTTATTTTTGATGACCTTGACAGTTTT |
| | GAGGAGTACTGGTTAGATATAGGGTAATGGTTTTTAAAGTATATTTGTCATGATTTATACTGGGGTAAGG |
| | GTTTGGGGAGGAAGCCCATGGGGTAAAGTACTGTTCTCATCACATCATATCAAGGTTATATACCATCAAT |
| | ATTGCCACAGATGTTACTTAGCCTTTTAATATTTCTCTAATTTAGTGTATATGCAATGATAGTTCTCTGA |
| | TTTCTGAGATTGAGTTTCTCATGTGTAATGATTATTTAGAGTTTCTCTTTCATCTGTTCAAATTTTTGTC |
| | TAGTTTTATTTTTTACTGATTTGTAAGACTTCTTTTTATAATCTGCATATTACAATTCTCTTTACTGGGG |
| | TGTTGCAAATATTTTCTGTCATTCTATGGCCTGACTTTTCTTAATGGTTTTTTAATTTTAAAAATAAGTC |
| | TTAATATTCATGCAATCTAATTAACAATCTTTTCTTTGTGGTTAGGACTTTGAGTCATAAGAAATTTTTC |
| | TCTACACTGAAGTCATGATGGCATGCTTCTATATTATTTTCTAAAAGATTTAAAGTTTTGCCTTCTCCAT |
| | TTAGACTTATAATTCACTGGAATTTTTTTGTGTGTATGGTATGACATATGGGTTCCCTTTTATTTTTTAC |
| | ATATAAATATATTTCCCTGTTTTTCTAAAAAAGAAAAAGATCATCATTTTCCCATTGTAAAATGCCATAT |
| | TTTTTTCATAGGTCACTTACATATATCAATGGGTCTGTTTCTGAGCTCTACTCTATTTTATCAGCCTCAC |
| | TGTCTATCCCCACACATCTCATGCTTTGCTCTAAATCTTGATATTTAGTGGAACATTCTTTCCCATTTTG |
| | TTCTACAAGAATATTTTTGTTATTGTCTTTTGGGCTTCTATATACATTTTAGAATGAGGTTGGCAAGTTA |
| | ACAAACAGCTTTTTTGGGGTGAACATATTGACTACAAATTTATGTGGAAAGAAAGTATACCTTCACAATA |
| | TTAAGTCTTTTAGTTCATGAATATAGTATGTCTCTCCGTTTCTGCATTAACTTAGACATTCATTAATTTC |
| | TCTCACAATTTATAAGTTTATTTAGATCT |
| SEQ ID | AAGAGGCATCAGGTAGTGAGTGACAAGTGAGCTTGCTTTCTAGTTTCCACCATGTGAGAGTGTAGTACCT |
| NO: 9 | AGACTTAGTGTCCTGGACTGAGGCTCCAGAACTGAGAGAAATACATTTCTGTTCTTTATAAATTTCCCAG |
| (DHFR- | AGTTCAGGATAAAATGGATGAAGACCTTGCCTTTACTGGGAGCAGTCATGTGGGATGCTGTCTTCGTATC |
| Reverse) | CCCTTTCTCCACTCACTTCACCGGTTTTTTAGGTCTGTTTAAATCAGGAAACTTAGTTGTCATGCTTATA |
| | AAAGAAATAGCTACAGATTTCTTAAAATACTAAGCTCTCTTTATAGTTTTCTTAAAATGTTAATTGAAGA |
| | GTAATGCCTGTCAAGAAGGCGGACATATTTTGTATTTGTGTTTCAGAGGCTGAGGTCCTTTTTGACATTT |
| | TGATTTTTATTTCCTTCTGAGTGCTATGATAGTATGACTTCTCTCCAGTGACAATGAAGATTTATCTTTT |
| | GAGTCTGTTTCTGTTGTGCTTTAGTGATAGGGCCTTTTATAATGAATATTCTTGTTTTATCATTCTAGGA |
| | ATTGTCCTTGTATCATGTCTTGGTGATTTATTTATTTTGTTTACTTTTTGGACTATTCTATTTTTGCAAG |
| | TTAATGTTTTAGGTTAATGATATGCACAATATGATTTTCTTATTATTCATGTCTTTGATGTTTATGTTTT |
| | TCTACTTTTAGTAAGATTTCATCAGATTTGTCATTCCACTTTATAAAGGATTTATACTAGTAGCATATTT |
| | CTTAATTTCTAAGAACTCTTTCTTGGCATCTGAATATTCTTTTTGTGTAGCACAGTCTAGTCTTTTATAA |
| | AATTTCTTTGATTTCTTTAAGATTATCAAAAACATCACACTGAGGCAGGGCACATACCTACAATACTAAC |
| | ACTCAGAAGACTAGAAACAGAAGCTCTAGAGTTTCAGACAAGCTTAGGATTCCTAGCAAGAACCTATAGA |
| | AACTTAGTTGAAAGTTATCTCTTGTTTTCATTATATTTGAATTCATATTTTCTGTGCCACATTTTCAAAA |
| | CCTCAAATCTATTGGAAGAAGGACTCTTCCAAAATTCAAGTGTTATGTTTGGTGTGGTGGTTCATGCCTT |
| | TAATTCCAGCACGTGGGAGGCAAAGGTAAGTGAGTCTCTCTGAGTTCAAGGCCAACCTGAACTATGTGAT |
| | GAATTCCATGCCCTGTCTCAAAAAAATATTTTAGTAATAATAGAGTATTTAGTTTCATGAGCTGATTGGT |
| | CATTTGCTCATTCTTAAAGCAGGTGCTGCCCAAGGAAACCAGAGTTGCCATGGTTTTCTGTTTGGTTTTG |
| | GGAGGGGATCATTATTGTCCCCTGGCATTAGCTGAGGAGCCAGCTCCACGAGGACTGCTTGAGAAGACCA |
| | ACCTTTCAAAGGAAGTTAAGATGCTATCAGTAAAATAAATCCTTTACGATATAGGATATCCTAAAAGGAA |
| | CATAATACTTTGACACAGGACCTGGCACAAAGGTTTTCTTTGGCTGGTTTCAGGCCAGTGTTTAGATGCT |
| | GGTAGTGTCCAGTTACACATAAGGGCAGAGCAGCCACAAAAATTCCAAGACAATTGCACTTCTTTAAAAA |
| | AAAAAGGAAAAAAGATTTATTTTATTTTTATATGCATAAATATTTGCCTGAATATATATATATATATATA |
| | TATATATATATATATATATATATATATAGTGTTCCTGGTTCCCATGGAAGCCAAAAGAGGGCATTGGAGG |
| | CTCCTAGAACTGGAACTTAAAACTAATTTTGGCCAGGTGTTGGTGGTGCATGCCTTTAATCCCAGGACTC |
| | GGGAGGCAAAGGCAGGCAGATCTCTGTGAGTTCGAGGCCAGCCTGGTCTCCAGAGCGAGTGCCAGGATAG |
| | GCTCCAAAGCTACACAGAGAAACCCTGTCTTGAAAAAAAACCTAAAAAACAAAAAACTAGTTTTGATGAG |
| | GGAGAGGGAGAAGGGATTGACATGTAAAGCAAGCTTCTTCCTAATTTGAACTAATAATAATAATAAAAAA |
| | ACTAGTTTTGAGTCATTTTATGGGCTTTTAAGACTATTTACTAAGTCTCATCAGAAATAGCAAGCTGGGG |
| | TATAAGCTACCTTGTAGCTTTGGTGGTTTGAATGAAAATGGCCCCATAGGCTCAGAGTGAGTGGAGGTGT |
| | GGCCTTGTTGGAAGAAGTGTGTCAATCTGATATAGAACTCTCATCTACTTCTCCAGCACCATGTCTGCCT |
| | GTGGCGCACCATACTTCCTGCAATCATGACAATGGATTAAACCTCTGAACTGTAAGCTGGACCCAATAAT |
| | TGCTTTCCTTTATAAGAGTTTCTGTGGTCATGGTGTCTCTTCACA |
| SEQ ID | GAATTCTACAAGCCTTTGTGAATATTTTTTCCTTCTCACTTGGCAAATACAATTCCTGAGATCAATAACC |
| NO: 10 | TCGTCTTTTTAATTTTTTCCTCGTCTTTTTAACTATTTATAAAATATTGAATTATAAAATATGTAATTAT |
| (Apo-B- | AAATACTTTAATTATAAAATATGTAATTATAAATACTTTAATTATAAAATATGTAATTATAAATACTTTA |
| Reverse) | TAAAATATGTAATTATAAAATATGTAATTATAAACATTTTAATTATAAAATATGTAATTATAAACATTTT |
| | AATTATAAAATATGTAATTATAAACATTTTAATTATAAAATATTTAATTATAAACATTTTAATTATAAAA |
| | TATTTAATTATAAATATTTTAATTATAAAATATTTAATTATAAATATTTTAATTATAAAATATTTAATTA |
| | TAAATATTTTAATTATAAATATTTTAATTATAAAATATTTAATTATAAAAACACAATTACCTCATCTTTT |
| | TGAATTC |
| SEQ ID | GAATTCAAACAACTCAATAGCAAGAAACCAAAAATCCCAATTAAAAAATGGGCAAAGGAACTGAATAGAC |
| NO: 11 | ATTTTTCCAAAGAAGACATGCAACTGGCCAAGAGATATATGAAAAAATTCTCAACATTACTAATCATCAA |
| (CPSi- | AAAATGCAAATTAAAACTGCAATGAGCTATCACTTTACACCTGTCAGATGGCTATTATCAAAAAGACAAA |
| Reverse) | AAATTATAAATGTTGGTGAGGATGTGGAGAAAAGGAAACCATTGTACACTGTTGATGAGAATGTAATTAG |
| | TATGGCCATATGGAAAGCTGTATGGAGCTCCTCAAAAGACTAAAAGTAGAATTATATGATCCTGCAATCT |
| | CACTTCTGAGATAGATCCGAAGGATTCGAAATCAGTACATTGAAGAGATAGTTGCACTCTCATGTTCATT |
| | GCTGCACTATTTACAATAGCCAATATATGGAATCAACAACCTAAGGGTCCATCAGTGGATTAATGGATAA |
| | GGAAAATGTGGTATATATACCCAATGGAATAGTATTCAGCCTTAACAAAGAATGAGATTCTGTTATTTGT |
| | TGCAACATGGATGAACCTGTAAGACACCATGCTAAGCAAGGATAAGCCAGCACAGAAAGCCAAATGCTGC |
| | ATGATCTCATTTATTACGTGGAGTCTAAAGCAATTGAACTCATAGAAGCAGAGTGTGTAATGGTGATTAC |
| | CAGAGGCTGGGAAGTGGGGGAATGAAAAGATGTTAGTCAAAGAGTACAAAGTTTCAGTTAGGCAGGAAGA |
| | ATAAGTTTTTAAAGATCTATGGTACATCATGGTGACTATAGTTAATAATAATAATGTATGTTTTGAAATT |
| | GCTAAAAGTAAATTTCAAATGTTCTCACTACAAAAAATGTTAAGTATTTGAGATGATAAAATGTTAATTG |
| | GCTTGATTTAATCAGTGTACATTGTGTATATATATATCATAACATCACTTTATACCCCATAAATATATAC |
| | AATAACAAATTTTAAAAAGTAAATTTAAAGCTGTCATTTGATAGCAGATTTAGTTTTTTAAAAATGTATA |
| | AATCCTAGCATGGAAAAAATAGCAAAGGGAGAAGGTAAAGGGAGTTAAATTTATTCATTACTTACCATGA |
| | TCCAGAGGACACATTAGTTACTTTACATTTGTTGTGTTATTTAAAAATTCTAAATGGGAGCTATTATTAT |
| | CACTTTTAAAGAAAAATTAGTACATCAAGGAGAATGGGATCCGAATTC |
| SEQ ID | GAATTCCTGGGTTTGGAGTGAGCAGGGGACTGGCACTGGTTAACTAAAGTAACAGGAGTTTCTTCCTCAC |
| NO: 12 | CTTCCTCCCAACTCCCCAGTCCTTATGCCTCTTTGTGTACTTAACTGCTACCTTTGACCTCTTGTATCTC |
| (C-Myc- | CTTTGCCTACCTCTCACCTTCTCACCTGCCTTCTGCCATTCCTTCTAACTGTCAACTCTGGCTGTTATCA |
| Reverse) | TAGTCTCCAGGAAGCATCTTAGAACCTCCAAGAACTTAAAGACCTTAAGGCCCCCAGACCCATTTCAACA |
| | GAGGAAAACTCTTGCCTTTCTTAAGACCGCCCTCTGAAGGGGCAATTGATGAAAACAAACAGGGATGGTG |
| | GTGGTTGGGAGGGGGAAGAAACGAAACAAAACATTCACAACTTAAGATTTGGCTCAATGATATATTTGCC |
| | AGTTATTTTATTTTTTCTAAAAACAATAGAAAAAAATCAACTTTAAAAAGCAAAATGTACTTAAATAAAA |
| | AAAATTAGGGTTTATAGTACCTATAATACTAGGTACTATATACTAGGATTGAAATTCTGTGTAACTGCTA |
| | TAAACGTTTTATTAAAGTTATTTACATTTAATGGCAATATTTACAGAGAAACATTGTGTAAATCTTAAAA |
| | TTTTTTAAAAACAATTCTTAAATACAAATCTGTTAAAGAAAAAAAAAAGATGGTAAGCATAAAAAAGTTC |
| | TTTTATGCCCAAAGTCCAATTTGAGGCAGTTTACATTATGGCTAAATCTTTCAGTCTCAAGACTCAGCCA |
| | AGGTTGTGAGGTTGCATTTGATCATGCATTTGAAACAAGTTCATAGGTGATTGCTCAGGACATTTCTGTT |
| | AGAAGGAATCGTTTTCCTTACTTTTCCTTACGCACAAGAGTTCCGTAGCTGTTCAAGTTTGTGTTTCAAC |
| | TGTTCTCGTCGTTTCCGCAACAAGTCCTCTTCAGAAATGAGCTTTTGCTCCTCTGCTTGGACGGACAGGA |
| | TGTATGCTGTGGCTTTTTTAAGGATAACTACCTTGGGGGCCTTTTCATTGTTTTCCAACTCCGGGATCTG |
| | GTCACGCAGGGCAAAAAAGCTCCGTTTTAGCTCGTTCCTCCTCTGGCGCTCCAAGACGTTGTGTGTTCGC |
| | CTCTTGACATTCTCCTCGGTGTCCGAGGACCTGGGGCTGGTGCATTTTCGGTTGTTGCTGATCTGTCTCA |
| | GGACTCTGACACTGTCCAACTTGACCCTCTTGGCAGCAGGATAGTCCTTCCGAGTGGAGGGAGGCGCTGC |
| | GTAGTTGTGCTGATGTGTGGAGACGTGGCACCTCTTGAGGACCAGTGGGCTGTGAGGAGGTTTGCTGTGG |
| | CCTCCAGCAGAAGGTGATCCAGACTCTGACCTTTTGCCAGGAGCCTGCCTCTTTTCCACAGAAACAACAT |
| | CGGAATTC |
| SEQ ID | GAATTCGGAGGGAAGGAAGGGCGGGGCGCGCCGTGTTTTGGACAACTTGTCCGGAGCTGCCTAGAACTAA |
| NO: 13 | CCACTCATAAAACCTCCAAACACAGGCGGAGAGCGCGGGGGTGGACGGGCACGAAGCGTTGATCCTGTGC |
| (KSHV- | GTAAACAAAAATACAGATGTAGAGCTAAAGCAGGGTGGGTTGTGACAGTTTTCCAGGGAAAGCCTGGATG |
| Reverse) | TAGGAAAATAGACACACATGTGGCGTCCGTGAATACGTTAGACATCTGTCAAGGCGGGATAGCGACAGTA |
| | GGGCGGACGGCGGGTCGGAGACTAACTGGGCACTCTGCTCACCTCCCCTTTCCCTTTTTCAGACTTCCAC |
| | CCCAGCTGCAGAGATGGCGTCTAAGGACGTAGAAGAGGGTGTAGAGGGACCCATCTGCTGGATATGTAGG |
| | GAAGAGGTGGGGAACGAGGGCATACACCCCTGCGCCTGTACCGGAGAGCTGGATGTCGTCCACCCGCAGT |
| | GTTTAAGCACTTGGCTAACAGTGTCTCGAAACACGGCCTGTCAAATGTGTCGCGTTATATACCGCACGCG |
| | CACGCAGTGGCGTAGTCGCCTTAACCTGTGGCCGGAGATGGAGCGCCAAGAAATTTTTGAACTGTTTCTG |
| | CTGATGTCTGTGGTGGTGGCTGGGCTTGTCGGCGTGGCGTTGTGCACCTGGACGCTCCTGGTCATCCTAA |
| | CTGCTCCTGCGGGAACATTCTCCCCCGGGGCCGTGCTCGGTTTTCTCTGCTTCTTTGGGTTTTACCAAAT |
| | ATTTATTGTGTTTGCATTTGGCGGCATATGCCGCGTAAGTGGCACTGTGAGGGCATTATACGCGGCAAAT |
| | AACACCCGGGTGACCGTACTGCCATACCGACGGCCGCGCCGGCCAACCGCGAACGAAGATAACATCGAAT |
| | TGACGGTCCTTGTCGGACCCGCGGGCGGGACGGACGAGGAGCCCACGGACGAGTCATCTGAAGGAGACGT |
| | CGCCTCTGGAGACAAAGAACGTGACGGTTCATCCGGAGACGAGCCGGACGGGGGCCCGAACGACCGTGCG |
| | GGACTTAGGGGGACAGCGCGTACCGACCTATGCGCGCCCACAAAAAAGCCGGTGCGGAAAAATCATCCAA |
| | AAAACAACGGTTGAACAACCACTTGGGTGCACAGAGATATGTGACGTGACGCCCTGGGTGTTATGCACCG |
| | TGCAGGTTTTCGCAGCGTATACACCCCCCCCCCCGCGCACACGTAGGGCTTTACCCTCTGGCCTGTGCCC |
| | AAATCTGTCCTCATTAAAAACAAGTAGCTGTTGACTTTCAAACAACCCGCGTGCGATATGTTTTCCATCC |
| | GTGTCCTCTAGTTTTCGGGTGATTAAGCAAAGGGGGGATGGGTGCAACGACAAATGAGGGTGAGTGGAAC |
| | CTTTTACTCGGTAAACATTGTTTATTTCAAGAGCCTATGCTGGGACAGAATAGCCGGGGAAAAGTGGACG |
| | GAGAATAGACCAGCCTGAAACGTCGCCCAAATCGTAGGCCAATACTGCCCAAGTATGGAATGGTGACTAA |
| | GAAAAACTCTCTCTAAACAATGTGAATCCTCTGTGCCCTCTTTGAATAAATGCTGGAACGGATGATTCTT |
| | ATTTCCGTTCGCTGGAAGCGGGATATGCTTCCGCCTCATCAAATACCTATACTACATATACGACACGCCT |
| | TGTGTACAGTGGGTGACACTAACACGGTGACGTGCTACCTGTGTAAATTTCGGTGTGGTTTTTAATGATA |
| | TTGGATAACGATGTTTTTAAAGTTTTTTGACGCGTGGCTGTGCTTCTCATCATGGTGCATTTGGGGGTTA |
| | TATGCGCGTGCTTGCGACGGATGCGGTCCGCCGTACGTGTAGAACGTTTTAAACACCATTAATACTAGCA |
| | GGGGTTGTGTGCTTGTGACTGATACAAATCGCCATACATGGCAACAGGTTGTTGCCGGGGAGGGTGGCGC |
| | GCCGGGATCCCCCTCCGAATTC |
| SEQ ID | CCTGCAGGCAGCTGCGCGCTCGCTCGCTCACTGAGGCCGCCCGGGCAAAGCCCGGGCGTCGGGCGACCTT |
| NO: 14 | TGGTCGCCCGGCCTCAGTGAGCGAGCGAGCGCGCAGAGAGGGAGTGGCCAACTCCATCACTAGGGGTTCC |
| (vector | TGCGGCCGATCTACAGGTGCCACCCCCTCCACCTTGGACACAGGACGCTGTGGTTTCTGAGCCAGGTACA |
| with c- | ATGACTCCTTTCGGTAAGTGCAGTGGAAGCTGTACACTGCCCAGGCAAAGCGTCCGGGCAGCGTAGGCGG |
| Myc- | GACTCAGATCCCAGCCAGTGGACTTAGCCCCTGTTTGCTCCTCCGATAACTGGGGTGACCTTGGTTAATA |
| Reverse | TTCACCAGCAGCCTCCCCCGTTGCCCCTCTGGATCCACTGCTTAAATACGGACGAGGACAGGGCCCTGTC |
| S/MAR) | TCCTCAGCTTCAGGCACCACCACTGACCTGGGACAGTGAAGGTACCTCGCCACCGGATCCAAGAGGTAAG |
| | GGTTTAAGGGATGGTTGGTTGGTGGGGTATTAATGTTTAATTACCTGTTTTACAGGCCTGAAATCACTTG |
| | GTTTTAGGTTGGGCCACCATGTCCAGCAAAGGCTCCGTGGTTCTGGCCTACAGTGGCGGCCTGGACACCT |
| | CGTGCATCCTCGTGTGGCTGAAGGAACAAGGCTATGACGTCATTGCCTATCTGGCCAACATTGGCCAGAA |
| | GGAAGACTTCGAGGAAGCCAGGAAGAAGGCACTGAAGCTTGGGGCCAAAAAGGTGTTCATTGAGGATGTC |
| | AGCAGGGAGTTTGTGGAGGAGTTCATCTGGCCGGCCATCCAGTCCAGCGCACTGTATGAGGACCGCTACC |
| | TCCTGGGCACCTCTCTTGCCAGGCCCTGCATCGCCCGCAAACAAGTGGAAATCGCCCAGCGGGAGGGGGC |
| | CAAGTATGTGTCCCACGGCGCCACAGGAAAGGGGAACGATCAGGTCCGGTTTGAGCTCAGCTGCTACTCA |
| | CTGGCCCCCCAGATAAAGGTCATTGCTCCCTGGAGGATGCCTGAATTCTACAACCGGTTCAAGGGCCGCA |
| | ATGACCTGATGGAGTACGCAAAGCAACACGGGATTCCCATCCCGGTCACTCCCAAGAACCCGTGGAGCAT |
| | GGATGAGAACCTCATGCACATCAGCTACGAGGCTGGAATCCTGGAGAACCCCAAGAACCAAGCGCCTCCA |
| | GGTCTCTACACGAAGACCCAGGACCCAGCCAAAGCCCCCAACACCCCTGACATTCTCGAGATCGAGTTCA |
| | AAAAAGGGGTCCCTGTGAAGGTGACCAACGTCAAGGATGGCACCACCCACCAGACCTCCTTGGAGCTCTT |
| | CATGTACCTGAACGAAGTCGCGGGCAAGCATGGCGTGGGCCGTATTGACATCGTGGAGAACCGCTTCATT |
| | GGAATGAAGTCCCGAGGTATCTACGAGACCCCAGCAGGCACCATCCTTTACCATGCTCATTTAGACATCG |
| | AGGCCTTCACCATGGACCGGGAAGTGCGCAAAATCAAACAAGGCCTGGGCTTGAAATTTGCTGAGCTGGT |
| | GTATACCGGTTTCTGGCACAGCCCTGAGTGTGAATTTGTCCGCCACTGCATCGCCAAGTCCCAGGAGCGA |
| | GTGGAAGGGAAAGTGCAGGTGTCCGTCCTCAAGGGCCAGGTGTACATCCTCGGCCGGGAGTCCCCACTGT |
| | CTCTCTACAATGAGGAGCTGGTGAGCATGAACGTGCAGGGTGATTATGAGCCAACTGATGCCACCGGGTT |
| | CATCAACATCAATTCCCTCAGGCTGAAGGAATATCATCGTCTCCAGAGCAAGGTCACTGCCAAATAGCGA |
| | AGCGGCCGCGACTCTAGGAATTCCTGGGTTTGGAGTGAGCAGGGGACTGGCACTGGTTAACTAAAGTAAC |
| | AGGAGTTTCTTCCTCACCTTCCTCCCAACTCCCCAGTCCTTATGCCTCTTTGTGTACTTAACTGCTACCT |
| | TTGACCTCTTGTATCTCCTTTGCCTACCTCTCACCTTCTCACCTGCCTTCTGCCATTCCTTCTAACTGTC |
| | AACTCTGGCTGTTATCATAGTCTCCAGGAAGCATCTTAGAACCTCCAAGAACTTAAAGACCTTAAGGCCC |
| | CCAGACCCATTTCAACAGAGGAAAACTCTTGCCTTTCTTAAGACCGCCCTCTGAAGGGGCAATTGATGAA |
| | AACAAACAGGGATGGTGGTGGTTGGGAGGGGGAAGAAACGAAACAAAACATTCACAACTTAAGATTTGGC |
| | TCAATGATATATTTGCCAGTTATTTTATTTTTTCTAAAAACAATAGAAAAAAATCAACTTTAAAAAGCAA |
| | AATGTACTTAAATAAAAAAAATTAGGGTTTATAGTACCTATAATACTAGGTACTATATACTAGGATTGAA |
| | ATTCTGTGTAACTGCTATAAACGTTTTATTAAAGTTATTTACATTTAATGGCAATATTTACAGAGAAACA |
| | TTGTGTAAATCTTAAAATTTTTTAAAAACAATTCTTAAATACAAATCTGTTAAAGAAAAAAAAAAGATGG |
| | TAAGCATAAAAAAGTTCTTTTATGCCCAAAGTCCAATTTGAGGCAGTTTACATTATGGCTAAATCTTTCA |
| | GTCTCAAGACTCAGCCAAGGTTGTGAGGTTGCATTTGATCATGCATTTGAAACAAGTTCATAGGTGATTG |
| | CTCAGGACATTTCTGTTAGAAGGAATCGTTTTCCTTACTTTTCCTTACGCACAAGAGTTCCGTAGCTGTT |
| | CAAGTTTGTGTTTCAACTGTTCTCGTCGTTTCCGCAACAAGTCCTCTTCAGAAATGAGCTTTTGCTCCTC |
| | TGCTTGGACGGACAGGATGTATGCTGTGGCTTTTTTAAGGATAACTACCTTGGGGGCCTTTTCATTGTTT |
| | TCCAACTCCGGGATCTGGTCACGCAGGGCAAAAAAGCTCCGTTTTAGCTCGTTCCTCCTCTGGCGCTCCA |
| | AGACGTTGTGTGTTCGCCTCTTGACATTCTCCTCGGTGTCCGAGGACCTGGGGCTGGTGCATTTTCGGTT |
| | GTTGCTGATCTGTCTCAGGACTCTGACACTGTCCAACTTGACCCTCTTGGCAGCAGGATAGTCCTTCCGA |
| | GTGGAGGGAGGCGCTGCGTAGTTGTGCTGATGTGTGGAGACGTGGCACCTCTTGAGGACCAGTGGGCTGT |
| | GAGGAGGTTTGCTGTGGCCTCCAGCAGAAGGTGATCCAGACTCTGACCTTTTGCCAGGAGCCTGCCTCTT |
| | TTCCACAGAAACAACATCGGAATTCCTGTGCCTTCTAGTTGCCAGCCATCTGTTGTTTGCCCCTCCCCCG |
| | TGCCTTCCTTGACCCTGGAAGGTGCCACTCCCACTGTCCTTTCCTAATAAAATGAGGAAATTGCATCGCA |
| | TTGTCTGAGTAGGTGTCATTCTATTCTGGGGGGTGGGGTGGGGCAGGACAGCAAGGGGGAGGATTGGGAA |
| | GACAATAGCAGGCATGCTGGGGATGCGGTGGGCTCTATGGAGGAACCCCTAGTGATGGAGTTGGCCACTC |
| | CCTCTCTGCGCGCTCGCTCGCTCACTGAGGCCGGGCGACCAAAGGTCGCCCGACGCCCGGGCTTTGCCCG |
| | GGCGGCCTCAGTGAGCGAGCGAGCGCGCAGCTGCCTGCAG |
| SEQ ID | >sp\|P00966\|ASSY_HUMAN Argininosuccinate synthase |
| NO: 15 | MSSKGSVVLAYSGGLDTSCILVWLKEQGYDVIAYLANIGQKEDFEEARKKALKLGAKKVFIEDVSREFVE |
| (ASS1) | EFIWPAIQSSALYEDRYLLGTSLARPCIARKQVEIAQREGAKYVSHGATGKGNDQVRFELSCYSLAPQIK |
| | VIAPWRMPEFYNRFKGRNDLMEYAKQHGIPIPVTPKNPWSMDENLMHISYEAGILENPKNQAPPGLYTKT |
| | QDPAKAPNTPDILEIEFKKGVPVKVTNVKDGTTHQTSLELFMYLNEVAGKHGVGRIDIVENRFIGMKSRG |
| | IYETPAGTILYHAHLDIEAFTMDREVRKIKQGLGLKFAELVYTGFWHSPECEFVRHCIAKSQERVEGKVQ |
| | VSVLKGQVYILGRESPLSLYNEELVSMNVQGDYEPTDATGFININSLRLKEYHRLQSKVTAK |
| SEQ ID | CCTGCAGGCAGCTGCGCGCTCGCTCGCTCACTGAGGCCGCCCGGGCAAAGCCCGGGCGTCGGGCGACCTT |
| NO: 16 | TGGTCGCCCGGCCTCAGTGAGCGAGCGAGCGCGCAGAGAGGGAGTGGCCAACTCCATCACTAGGGGTTCC |
| (5′ITR) | T |
| SEQ ID | AGGAACCCCTAGTGATGGAGTTGGCCACTCCCTCTCTGCGCGCTCGCTCGCTCACTGAGGCCGGGCGACC |
| NO: 17 | AAAGGTCGCCCGACGCCCGGGCTTTGCCCGGGCGGCCTCAGTGAGCGAGCGAGCGCGCAGCTGCCTGCAG |
| (3′ITR) | G |
| SEQ ID | CTGTGCCTTCTAGTTGCCAGCCATCTGTTGTTTGCCCCTCCCCCGTGCCTTCCTTGACCCTGGAAGGTGC |
| NO: 18 | CACTCCCACTGTCCTTTCCTAATAAAATGAGGAAATTGCATCGCATTGTCTGAGTAGGTGTCATTCTATT |
| (BGHpA) | CTGGGGGGTGGGGTGGGGCAGGACAGCAAGGGGGAGGATTGGGAAGACAATAGCAGGCATGCTGGGGATG |
| | CGGTGGGCTCTATGG |
| SEQ ID | GGTGCCACCCCCTCCACCTTGGACACAGGACGCTGTGGTTTCTGAGCCAGGTACAATGACTCCTTTCGGT |
| NO: 19 | AAGTGCAGTGGAAGCTGTACACTGCCCAGGCAAAGCGTCCGGGCAGCGTAGGCGGGACTCAGATCCCAGC |
| (AAT | CAGTGGACTTAGCCCCTGTTTGCTCCTCCGATAACTGGGGTGACCTTGGTTAATATTCACCAGCAGCCTC |
| promoter) | CCCCGTTGCCCCTCTGGATCCACTGCTTAAATACGGACGAGGACAGGGCCCTGTCTCCTCAGCTTCAGGC |
| | ACCACCACTGACCTGGGACAGTGAA |
| SEQ ID | ATGTCCAGCAAAGGCTCCGTGGTTCTGGCCTACAGTGGCGGCCTGGACACCTCGTGCATCCTCGTGTGGC |
| NO: 20 | TGAAGGAACAAGGCTATGACGTCATTGCCTATCTGGCCAACATTGGCCAGAAGGAAGACTTCGAGGAAGC |
| (ASS1 | CAGGAAGAAGGCACTGAAGCTTGGGGCCAAAAAGGTGTTCATTGAGGATGTCAGCAGGGAGTTTGTGGAG |
| transgene) | GAGTTCATCTGGCCGGCCATCCAGTCCAGCGCACTGTATGAGGACCGCTACCTCCTGGGCACCTCTCTTG |
| | CCAGGCCCTGCATCGCCCGCAAACAAGTGGAAATCGCCCAGCGGGAGGGGGCCAAGTATGTGTCCCACGG |
| | CGCCACAGGAAAGGGGAACGATCAGGTCCGGTTTGAGCTCAGCTGCTACTCACTGGCCCCCCAGATAAAG |
| | GTCATTGCTCCCTGGAGGATGCCTGAATTCTACAACCGGTTCAAGGGCCGCAATGACCTGATGGAGTACG |
| | CAAAGCAACACGGGATTCCCATCCCGGTCACTCCCAAGAACCCGTGGAGCATGGATGAGAACCTCATGCA |
| | CATCAGCTACGAGGCTGGAATCCTGGAGAACCCCAAGAACCAAGCGCCTCCAGGTCTCTACACGAAGACC |
| | CAGGACCCAGCCAAAGCCCCCAACACCCCTGACATTCTCGAGATCGAGTTCAAAAAAGGGGTCCCTGTGA |
| | AGGTGACCAACGTCAAGGATGGCACCACCCACCAGACCTCCTTGGAGCTCTTCATGTACCTGAACGAAGT |
| | CGCGGGCAAGCATGGCGTGGGCCGTATTGACATCGTGGAGAACCGCTTCATTGGAATGAAGTCCCGAGGT |
| | ATCTACGAGACCCCAGCAGGCACCATCCTTTACCATGCTCATTTAGACATCGAGGCCTTCACCATGGACC |
| | GGGAAGTGCGCAAAATCAAACAAGGCCTGGGCTTGAAATTTGCTGAGCTGGTGTATACCGGTTTCTGGCA |
| | CAGCCCTGAGTGTGAATTTGTCCGCCACTGCATCGCCAAGTCCCAGGAGCGAGTGGAAGGGAAAGTGCAG |
| | GTGTCCGTCCTCAAGGGCCAGGTGTACATCCTCGGCCGGGAGTCCCCACTGTCTCTCTACAATGAGGAGC |
| | TGGTGAGCATGAACGTGCAGGGTGATTATGAGCCAACTGATGCCACCGGGTTCATCAACATCAATTCCCT |
| | CAGGCTGAAGGAATATCATCGTCTCCAGAGCAAGGTCACTGCCAAATAGCGA |
| SEQ ID | GGATCCAAGAGGTAAGGGTTTAAGGGATGGTTGGTTGGTGGGGTATTAATGTTTAATTACCTGTTTTACA |
| NO: 21 | GGCCTGAAATCACTTGGTTTTAGGTTGGGCCACC |
| (MVM | |
| intron) | |
| SEQ ID | CCTGCAGGCAGCTGCGCGCTCGCTCGCTCACTGAGGCCGCCCGGGCAAAGCCCGGGCGTCGGGCGACCTT |
| NO: 22 | TGGTCGCCCGGCCTCAGTGAGCGAGCGAGCGCGCAGAGAGGGAGTGGCCAACTCCATCACTAGGGGTTCC |
| (vector | TGCGGCCGATCTACAGGTGCCACCCCCTCCACCTTGGACACAGGACGCTGTGGTTTCTGAGCCAGGTACA |
| with IFN- | ATGACTCCTTTCGGTAAGTGCAGTGGAAGCTGTACACTGCCCAGGCAAAGCGTCCGGGCAGCGTAGGCGG |
| β-Forward) | GACTCAGATCCCAGCCAGTGGACTTAGCCCCTGTTTGCTCCTCCGATAACTGGGGTGACCTTGGTTAATA |
| | TTCACCAGCAGCCTCCCCCGTTGCCCCTCTGGATCCACTGCTTAAATACGGACGAGGACAGGGCCCTGTC |
| | TCCTCAGCTTCAGGCACCACCACTGACCTGGGACAGTGAAGGTACCTCGCCACCGGATCCAAGAGGTAAG |
| | GGTTTAAGGGATGGTTGGTTGGTGGGGTATTAATGTTTAATTACCTGTTTTACAGGCCTGAAATCACTTG |
| | GTTTTAGGTTGGGCCACCATGTCCAGCAAAGGCTCCGTGGTTCTGGCCTACAGTGGCGGCCTGGACACCT |
| | CGTGCATCCTCGTGTGGCTGAAGGAACAAGGCTATGACGTCATTGCCTATCTGGCCAACATTGGCCAGAA |
| | GGAAGACTTCGAGGAAGCCAGGAAGAAGGCACTGAAGCTTGGGGCCAAAAAGGTGTTCATTGAGGATGTC |
| | AGCAGGGAGTTTGTGGAGGAGTTCATCTGGCCGGCCATCCAGTCCAGCGCACTGTATGAGGACCGCTACC |
| | TCCTGGGCACCTCTCTTGCCAGGCCCTGCATCGCCCGCAAACAAGTGGAAATCGCCCAGCGGGAGGGGGC |
| | CAAGTATGTGTCCCACGGCGCCACAGGAAAGGGGAACGATCAGGTCCGGTTTGAGCTCAGCTGCTACTCA |
| | CTGGCCCCCCAGATAAAGGTCATTGCTCCCTGGAGGATGCCTGAATTCTACAACCGGTTCAAGGGCCGCA |
| | ATGACCTGATGGAGTACGCAAAGCAACACGGGATTCCCATCCCGGTCACTCCCAAGAACCCGTGGAGCAT |
| | GGATGAGAACCTCATGCACATCAGCTACGAGGCTGGAATCCTGGAGAACCCCAAGAACCAAGCGCCTCCA |
| | GGTCTCTACACGAAGACCCAGGACCCAGCCAAAGCCCCCAACACCCCTGACATTCTCGAGATCGAGTTCA |
| | AAAAAGGGGTCCCTGTGAAGGTGACCAACGTCAAGGATGGCACCACCCACCAGACCTCCTTGGAGCTCTT |
| | CATGTACCTGAACGAAGTCGCGGGCAAGCATGGCGTGGGCCGTATTGACATCGTGGAGAACCGCTTCATT |
| | GGAATGAAGTCCCGAGGTATCTACGAGACCCCAGCAGGCACCATCCTTTACCATGCTCATTTAGACATCG |
| | AGGCCTTCACCATGGACCGGGAAGTGCGCAAAATCAAACAAGGCCTGGGCTTGAAATTTGCTGAGCTGGT |
| | GTATACCGGTTTCTGGCACAGCCCTGAGTGTGAATTTGTCCGCCACTGCATCGCCAAGTCCCAGGAGCGA |
| | GTGGAAGGGAAAGTGCAGGTGTCCGTCCTCAAGGGCCAGGTGTACATCCTCGGCCGGGAGTCCCCACTGT |
| | CTCTCTACAATGAGGAGCTGGTGAGCATGAACGTGCAGGGTGATTATGAGCCAACTGATGCCACCGGGTT |
| | CATCAACATCAATTCCCTCAGGCTGAAGGAATATCATCGTCTCCAGAGCAAGGTCACTGCCAAATAGCGA |
| | AGCGGCCGCGACTCTAGAGATCTAAATAAACTTATAAATTGTGAGAGAAATTAATGAATGTCTAAGTTAA |
| | TGCAGAAACGGAGAGACATACTATATTCATGAACTAAAAGACTTAATATTGTGAAGGTATACTTTCTTTC |
| | CACATAAATTTGTAGTCAATATGTTCACCCCAAAAAAGCTGTTTGTTAACTTGCCAACCTCATTCTAAAA |
| | TGTATATAGAAGCCCAAAAGACAATAACAAAAATATTCTTGTAGAACAAAATGGGAAAGAATGTTCCACT |
| | AAATATCAAGATTTAGAGCAAAGCATGAGATGTGTGGGGATAGACAGTGAGGCTGATAAAATAGAGTAGA |
| | GCTCAGAAACAGACCCATTGATATATGTAAGTGACCTATGAAAAAAATATGGCATTTTACAATGGGAAAA |
| | TGATGATCTTTTTCTTTTTTAGAAAAACAGGGAAATATATTTATATGTAAAAAATAAAAGGGAACCCATA |
| | TGTCATACCATACACACAAAAAAATTCCAGTGAATTATAAGTCTAAATGGAGAAGGCAAAACTTTAAATC |
| | TTTTAGAAAATAATATAGAAGCATGCCATCATGACTTCAGTGTAGAGAAAAATTTCTTATGACTCAAAGT |
| | CCTAACCACAAAGAAAAGATTGTTAATTAGATTGCATGAATATTAAGACTTATTTTTAAAATTAAAAAAC |
| | CATTAAGAAAAGTCAGGCCATAGAATGACAGAAAATATTTGCAACACCCCAGTAAAGAGAATTGTAATAT |
| | GCAGATTATAAAAAGAAGTCTTACAAATCAGTAAAAAATAAAACTAGACAAAAATTTGAACAGATGAAAG |
| | AGAAACTCTAAATAATCATTACACATGAGAAACTCAATCTCAGAAATCAGAGAACTATCATTGCATATAC |
| | ACTAAATTAGAGAAATATTAAAAGGCTAAGTAACATCTGTGGCAATATTGATGGTATATAACCTTGATAT |
| | GATGTGATGAGAACAGTACTTTACCCCATGGGCTTCCTCCCCAAACCCTTACCCCAGTATAAATCATGAC |
| | AAATATACTTTAAAAACCATTACCCTATATCTAACCAGTACTCCTCAAAACTGTCAAGGTCATCAAAAAT |
| | AAGAAAAGTCTGAGGAACTGTCAAAACTAAGAGGAACCCAAGGAGACATGAGAATTATATGTAATGTGGC |
| | ATTCTGAATGAGATCCCAGAACAGAAAAAGAACAGTAGCTAAAAAACTAATGAAATATAAATAAAGTTTG |
| | AACTTTAGTTTTTTTTAAAAAAGAGTAGCATTAACACGGCAAAGCCATTTTCATATTTTTCTTGAACATT |
| | AAGTACAAGTCTATAATTAAAAATTTTTTAAATGTAGTCTGGAACATTGCCAGAAACAGAAGTACAACAG |
| | CTATCTGTGCTGTCGCCTAACTATCCATAGCTGATTGGTCTAAAATGAGATACATCAACGCTCCTCCATG |
| | TTTTTTGTTTTCTTTTTAAATGAAAAACTTTATTTTTTAAGAGGAGTTTCAGGTTCATAGCAAAATTGAG |
| | AGGAAGGTACATTCAAGCTGAGGAAGTTTTCCTCTATTCCTAGTTTACTGAGAGATTGCATCATGAATGG |
| | GTGTTAAATTTTGTCAAATGCTTTTTCTGTGTCTATCAATATGACCATGTGATTTTCTTCTTTAACCTGT |
| | TGATGGGACAAATTACGTTAATTGATTTTCAAACGTTGAACCACCCTTACATATCTGGAATAAATTCTAC |
| | TTGGTTGTGGTGTATATTTTTTGATACATTCTTGGATTCTTTTTGCTAATATTTTGTTGAAAATGTTTGT |
| | ATCTTTGTTCATGAGAGATATTGGTCTGTTGTTTTCTTTTCTTGTAATGTCATTTTCTAGTTCCGGTATT |
| | AAGGTAATGCTGGCCTAGTTGAATGATTTAGGAAGTATTCCCTCTGCTTCTGTCTTCTGAAAGAGATTGT |
| | AGAAAGTTGATACAATTTTTTTTTCTTTAAATATTTGATAGAATTCCTGTGCCTTCTAGTTGCCAGCCAT |
| | CTGTTGTTTGCCCCTCCCCCGTGCCTTCCTTGACCCTGGAAGGTGCCACTCCCACTGTCCTTTCCTAATA |
| | AAATGAGGAAATTGCATCGCATTGTCTGAGTAGGTGTCATTCTATTCTGGGGGGTGGGGTGGGGCAGGAC |
| | AGCAAGGGGGAGGATTGGGAAGACAATAGCAGGCATGCTGGGGATGCGGTGGGCTCTATGGAGGAACCCC |
| | TAGTGATGGAGTTGGCCACTCCCTCTCTGCGCGCTCGCTCGCTCACTGAGGCCGGGCGACCAAAGGTCGC |
| | CCGACGCCCGGGCTTTGCCCGGGCGGCCTCAGTGAGCGAGCGAGCGCGCAGCTGCCTGCAG |
| SEQ ID | CCTGCAGGCAGCTGCGCGCTCGCTCGCTCACTGAGGCCGCCCGGGCGTCGGGCGACCTTTGGTCGCCCGG |
| NO: 23 | CCTCAGTGAGCGAGCGAGCGCGCAGAGAGGGAGTGGCCAACTCCATCACTAGGGGTTCCT |
| (5′ITR) | |

The following examples are provided by way of illustration, and they are not intended to be limiting of the present invention.

### EXAMPLES

### Methods

A. Animals

Adults C57BL/6j males and females were obtained from The Jackson Laboratory stock number 000664 and were bred at the CIMA animal house. All mice were inoculated at 0 days of age and were group-housed in ventilated cages (maximum of 6 animals per cage) in a temperature-controlled room kept on a 12 h light-dark cycle until the end of the study. Food and water were provided ad libitum. All animal experiments were performed in strict accordance with the Animal Ethical Committee guidelines of the University of Navarra. The protocol was approved by the Animal Ethical Committee of the University of Navarra. Every effort was made to minimize the number of animals used and their suffering.

B. Study Design

Study design is summarized in Table 2 below.

N means “Number”, i.e. between 6 and 8 males or females. W means “Week”. «AAT-eGFP-S/MAR» refers to a vector according to the invention, and «AAT-eGFP» refers to the same vector but without the S/MAR sequences (it does not correspond to a vector according to the invention).

TABLE 2
Study design

| | | | AAV | | | | | |
|---|---|---|---|---|---|---|---|---|
| | | | Treatment | | Terminal | | | |
| Group | Strain | N | (AAVT) | Age of AAVT | Age | | Collections | Analysis |
| 1 | C57BL/6 | 6-8M/F | AAT-eGFP | Newborn | 1 | W | Term | Liver: |
| 2 | | 6-8M/F | AAT-eGFP | Newborn | 3 | W | Liver | Viral |
| 3 | | 6-8M/F | AAT-eGFP | Newborn | 6 | W | | genomes by |
| 4 | | 6-8M/F | AAT-eGFP | Newborn | 12 | W | | qPCR |
| 5 | | 6-8M/F | AAT-eGFP- | Newborn | 1 | W | | |
| | | | S/MAR | | | | | |
| 6 | | 6-8M/F | AAT-eGFP- | Newborn | 3 | W | | |
| | | | S/MAR | | | | | |
| 7 | | 6-8M/F | AAT-eGFP- | Newborn | 6 | W | | |
| | | | S/MAR | | | | | |
| 8 | | 6-8M/F | AAT-eGFP- | Newborn | 12 | W | | |
| | | | S/MAR | | | | | |

C. Statistical Analysis

Significant differences between two independent groups (S/MAR vs NO S/MAR) were analyzed using an unpaired t test. Tests were performance with a 95% of confidence using the non-parametric Mann-Whitney test. P<0.05 was considered significant. All analyses were performed using GraphPad Prism 7.0 for Windows. All graphs show the mean values±SEM.

D. Results

In Examples 1 to 4, the effect produced by 6 different S/MAR elements, sub-cloned in orientation forward and reverse, in an AAV virus genome has been studied. The created vectors carrying the S/MAR elements were used for the transduction of neonatal murine hepatocytes for the study of the effect of these sequences on the stability of the AAV viral genome in growing livers of C57BL/6j mice.

### Example 1: Generation and Sub-Cloning of the Different Expression Cassettes Containing the S/MAR Elements into Plasmids Containing the Genome of the SSAAV Vector

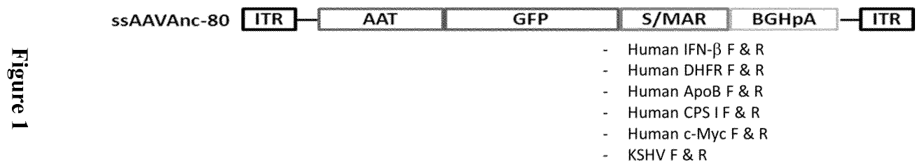

The construction of the recombinant plasmids was performed in the pAAV-MCS by the use of different restriction and ligation enzymes. To this, 6 S/MAR sequences were synthesized by the company ThermoFisher Scientific. Then the 6 S/MAR sequences were inserted in forward and reverse orientation between the open reading frame (ORF) of the reporter gene, the green fluorescence protein (GFP), and the polyadenylation signal under the control of the α-1-antitrypsin (AAT) promoter (FIG. 1). After the cloning, the plasmids were characterized by checking the correct restriction pattern and by sequencing. Subsequently, the plasmids were purified and multiplied successfully through the use of commercial miniprep and maxiprep kits.

Once this first phase of design and sub-cloning of the 6 S/MAR elements, in forward and reverse orientation, into AAV vectors was completed, a second phase of production of 12 different AAV viruses began.

### Example 2: Production of the 12 Different AAV Viruses Carrying the Constructs with 6 Different S/MAR Elements on AAV-Anc-80 Serotype Viral Particles

The production of the 12 AAV-Anc-80 serotype viral particles containing the 6 S/MAR sequences, in forward and reverse orientation, and the virus used as control without any S/MAR element was carried out by cell transfection of the different plasmids constructed in Example 1, which contain the AAV genome with S/MAR sequences, and the plasmids that contain the AAV rep and cap genes and the genes from the adenovirus that allows AAVs to complete its cycle and give rise to new viral particles in HEK-293 cells. Subsequently, the cells were harvest and the viruses were purified and titrated. These productions of the viruses with the S/MAR sequences had an average titer of 1×10¹² viral genomes (vg)/mL, which was enough to begin with in vivo AAV genomes prevalence studies.

### Example 3: In Vivo Evaluation on the Stability of the 12 Different AAV-Anc-80-S/MAR Viruses Generated in Neonates' Mice

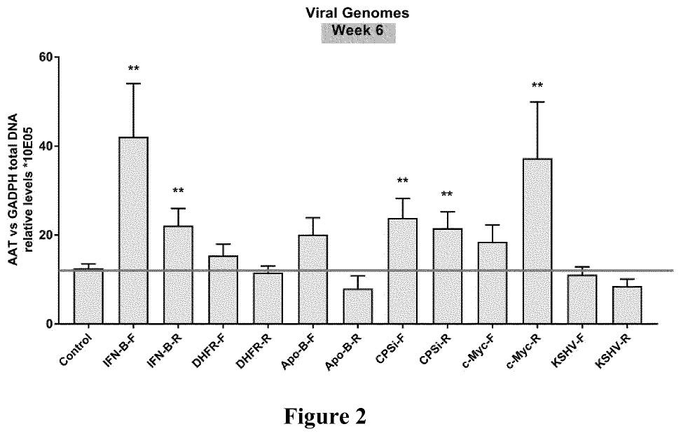

The 12 vectors containing the 6 S/MAR elements in forward and reverse orientation (ssAAV-Anc80-GFP-S/MAR) and the control virus without any S/MAR element (ssAAV-Anc80-GFP), produced as mentioned above, were inoculated intravenously to groups of 6-8 C57BL/6 mice 24 hours after birth at a dose of 5×10⁹ vg/g. Mice were sacrificed 1, 3 and 6 weeks after virus inoculation and liver samples were obtained. Obtained livers were processed to evaluate the presence of viral genomes on the hepatocytes. After quantification of the number of viral genomes, 6 weeks after virus inoculation, 5 out of 12 of the groups (IFN-β forward (IFN-β-F), IFN-β reverse (IFN-β-R), CPSI forward (CPSI-F), CPSI reverse (CPSI-R) and cMyc reverse (c-Myc-R) S/MAR sequences) of animals inoculated with the ssAAV-Anc80-GFP-S/MAR vectors have a significantly higher number of viral genomes than the livers of animals inoculated with ssAAV-Anc80-GFP (FIG. 2). (95% confidence level, two-tailed t test vs control; IFN-β-F p=0.003; IFN-β-R p=0.003; DHFR-F p=1; DHFR-R p=0.7; Apo-B-F p=0.1; Apo-B-R p=0.09; CPSI-F p=0.002; CPSI-R p=0.006; c-Myc-F p=0.3; c-Myc-R p=0.004; KSHV-F p=0.5; KSHV-R p=0.06).

IFN-β-Forward and c-Myc-reverse S/MAR sequences presents the higher amount of viral genomes at the 6 weeks after AAV inoculation.

### Example 4: Characterization of AAV Comprising IFN-β-F and c-Myc-R S/MAR Elements

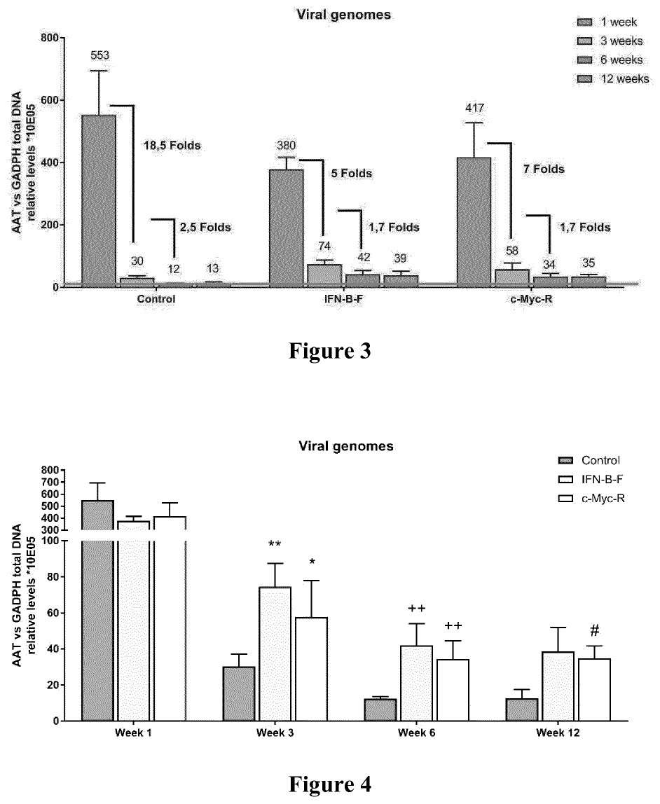

From the 5 SMAR sequences that presented a positive effect on AAV genome maintenance when the liver grows, vectors with the S/MAR sequences IFN-β-Forward and c-Myc-Reverse as the candidates with the best potential for the improvement of gene therapy in neonates with AAVs. As it was described previously, these two vectors containing the S/MAR elements IFN-β-Forward and c-Myc-Reverse and the control virus without any S/MAR element (ssAAV-Anc80-GFP) were inoculated intravenously to groups of 8-10 neonatal C57BL/6 mice at a dose of 5×10⁹ vg/g. The animals were sacrificed for liver collection at weeks 1, 3, 6 and 12 after AAV administration. Both, IFN-β-Forward and c-Myc-Reverse, shows a statistically similar infectivity to the control vector in the liver of the mice 1 week after inoculation (FIG. 4). However, the loss of viral genomes between week 1 and week 3 is more pronounced in the control vector, ssAAV-Anc80-GFP, than in the vectors cloned with the S/MAR elements IFN-β-Forward and c-Myc-Reverse. While the decrease in the number of viral genomes between week 1 and 3 in ssAAV-Anc80-GFP inoculated mice is of 18 folds (control), in the animals administered with the vectors containing IFN-β-Forward and c-Myc-Reverse S/MAR sequences the decrease is of 5 and 7 folds respectively (FIG. 3). The decrease on viral genomes presents in the liver between week 3 and week 6 is less marked than between week 1 and 3 in all the groups but still is a bit higher in the control group, ssAAV-Anc80-GFP (2.5 folds) in comparison with both ssAAV-Anc80-GFP-S/MAR viruses (1.7 folds) (FIG. 3). The number of viral genomes between week 6 and 12 is maintained constantly in all the groups studied. Interestingly, both AAVs with the S/MAR sequences (IFN-β-Forward and c-Myc-Reverse), maintain a significantly higher number of viral genomes than the control group 3 (95% confidence level, two-tailed t test vs control; IFN-β-F p=0.001; c-Myc-R p=0.04) and 6 weeks (95% confidence level, two-tailed t test vs control; IFN-β-F p=0.003; c-Myc-R p=0.001) after AAV inoculation. c-Myc-R S/MAR recombinant AAV presents significant differences even 12 weeks after AAV inoculation (95% confidence level, two-tailed t test vs control; p=0.04).

Therefore, the selected vectors, with IFN-β-Forward and the c-Myc-Reverse S/MAR elements have the capacity to decrease the magnitude of viral genomes loss over time when are inoculated intravenously in neonate mice.

### Example 5

### 5.1. Material and Methods

### 5.1.1 Animals

Adult males and females C57BL/6j (stock number 000664) and B6Ei.PAss1fold/fold/GrsrJ (Fold/Fold; stock number 006449) mice and their WT littermates were obtained from The Jackson Laboratory, and were bred at the CIMA animal house. All mice were group-housed in ventilated cages (maximum of 6 animals per cage) in a temperature-controlled room and were kept on a 12 h light-dark cycle until the end of the study. Food and water were provided ad libitum. Treatments with rAAV vectors were performed in male and female mice at the described age by intravenous injection under general anesthesia with Isoflurane (ISOVET. Braun). Liver samples were collected from euthanized mice for histological analysis and nucleic acid extraction. All animal experiments were performed in strict accordance with the Animal Ethical Committee guidelines of the University of Navarra. The protocol was approved by the Animal Ethical Committee of the University of Navarra (protocols 119-15, 070-17 and 072-17). Every effort was made to minimize the number of animals used and their suffering.

5.1.2. Construction of rAAV Vector Genomes

The construction of pAAV-AAT-eGFP and pAAV-AAT-eGFP-IFN-β-Forward was described in Example 1.

For the construction of pAAV-EAlbAAT-hASS1+INT, EA1bAAT promoter sequence (SEQ ID NO: 19) was synthetized by Invitrogen (thermo fisher scientific) in a pMS-RQ backbone. DNA promoter sequence was amplified by PCR.

The amplified fragment was then sub-cloned into the shuttle pAAV-MCS vector, that contains the AAV2 WT inverted terminal repeats (ITRs) (5′ITR, SEQ ID NO: 23; 3′ITR: SEQ ID NO:17), using the restriction enzyme NotI (New England Biolabs Inc.). Finally, the ASS1 WT+INT sequence synthetized by Invitrogen (Thermo Fisher Scientific) (ASS1 transgene, SEQ ID NO: 20 and MVM intron: SEQ ID NO: 21) was subcloned into the pAAV-EA1bAAT vector using the restriction enzymes PmlI, BamHI and NotI (New England Biolabs Inc.) to obtain the final vector pAAV-EAlbAAT-ASS1WT+INT.

For the construction of pAAV-EAlbAAT-hASS1+INT-IFN-β-Forward vector, IFN-β-Forward S/MAR sequence was obtained from plasmid pAAV-eGFP-IFN-β-Forward (cloned as described before) by MluI digestion (New England Biolabs Inc.). Thereafter it was subcloned into the pAAV-EAlbAAT-hASS1+INT plasmid using the restriction enzyme NotI and the DNA T4 ligase (New England Biolabs Inc.) to obtain the final vector pAAV-EAlbAAT-ASS1WT+INT-IFN-β-Forward.

All constructions were checked by restriction enzyme digestion and sequencing when PCR was used.

### 5.1.3. Preparation of AAV Vectors: Production, Purification and Titration.

rAAV serotype Anc80 vectors with AAV2 ITRs were produced by polyethyleneimine-mediated co-transfection in HEK-293T cells. Previous to cell transfection, each AAV shuttle vector plasmid, the helper/packaging plasmid (pKan-Anc80AAP-2) and an adenoviral helper plasmid (pDF6) were amplified using the Nucleobond extra maxi Kit (Macherey Nagel) according to the manufacturer's instructions. HEK-293T cells (CRL-3216. ATCC) were cultured in 150 mm plates in Dulbecco's modified Eagle's medium+1% Sodium pyruvate (DMEM. GIBCO) supplemented with 10% heat-inactivated fetal bovine serum (FBS. Gibco), 1% penicillin and streptomycin (Invitrogen) and placed the dishes in an incubator at 37° C. and 5% CO2 for 24 hours. Cells were then transfected with a different plasmid mix and polyethyleneimine (3:1). 72 hours later, cells were harvested, virus was purified by iodixanol (Optiprep. ATOM) gradient and concentrated using Amicon Ultra Centrifugal Filters-Ultracel 100K (Millipore) in a total volume of 1 ml. Titration of viral particles was done by qPCR using primers complementary to the AAT region.

5.1.4. Nucleic Acid Extraction and qPCR

To quantify viral genomes, total DNA was extracted from frozen livers using the Maxwell 16 LEV Blood DNA kit (Promega), according to the manufacturer's instructions. Total DNA was analyzed for the quantification of rAAV genomes by the amplification of AAT by qPCR and samples were normalized mGAPDH (glyceraldehyde-3-phosphate dehydrogenase). To analyze transgene expression, total RNA was isolated from frozen livers using the Maxwell 16 LEV Simply RNA tissue kit (Promega), according to the manufacturer's instructions. Extracted RNA was pretreated with Turbo DNA Free (Ambion) and retrotranscribed into cDNA using M-MLV reverse transcriptase (Invitrogen). Copies of human ASST in cDNA were quantified by qPCR using GoTaq qPCR Mastermix (Promega) in a CFX96 Real-Time Detection System (BioRad) and normalized to mouse histone.

### 5.1.5. Histological Analysis

Immunohistochemistry of eGFP in the livers was performed by the Morphology Facility of Centro de Investigación Médica Aplicada (CIMA; Pamplona. Spain) by the use of Anti-GFP antibody (AB6556. ABCAM). Quantification of the positive area in GFP was performed by Image acquisition using a slide scanner (Digital Pathology Slide Scanner Leica Aperio CS2) at a 40× magnification. Images were subsequently extracted to JPG sections. A set of FIJI plugins (a distribution of ImageJ; NIH, Bethesda, Md.) was developed by the Image Core Facility at CIMA to analyze the average positive area (%).

### 5.1.6. Statistical Analysis

Significant differences between two independent groups were analyzed using an unpaired t test. Tests were performance with a 95% of confidence using the non-parametric test of Mann-Whitney for the study of viral genomes and transgene expression. P<0.05 was considered significant. All analyses were performed using GraphPad Prism 7.0 for Windows. All graphs show the mean values±SEM.

### 5.2 Results

After the selection of the S/MAR sequence, IFN-β-Forward, as the S/MAR element with the highest potential for its optimization, the inventors has evaluated the short- and mid-term effect on viral genomes persistence, produced by this S/MAR element present in a rAAV vector genome when it is inoculated to infantile mice at different ages. Furthermore, the inventors has studied also the properties on viral genomes persistence provided by the IFN-β-Forward S/MAR sequence when it is located in a therapeutic vector for the treatment of CTLN1 when it is inoculated to infantile mice that mimic the signs and symptoms of this disease, the Fold/Fold mice.

### 5.2.1 Determination of the Most Efficient Time Point for the Inoculation of the S/MAR Vector

The construction of the recombinant plasmids carrying the reporter gene green fluorescent protein (eGFP) (FIG. 1) and the production of the virus were performed as described in examples 1 and 2. The titer of the used batches for the experiment was 5×10¹² viral genomes (vg)/ml.

Groups of 6-9 male/females C57BL/6 mice were inoculated intravenously with a dose of 5×10¹² vg/Kg of the vectors with and without the S/MAR sequence: AAV-AAT-eGFP (reference vector), AAV-AAT-eGFP-IFN-β-Forward (S/MAR vector)) at 2- and 3-weeks of age. Liver samples were collected 3 days after virus inoculation and at 6 weeks of life for the analysis of viral genomes by PCR and the percentage of transgene (GFP) positive hepatocytes by immunohistochemistry.

Evaluation of the number of viral genomes and the transduced cells was performed at the time points as described in Table 3 below.

TABLE 3
In vivo study design for the analysis of rAAV viral
genomes maintenance study at 2- and 3-weeks of age.

| | | | | | Termial Age: | | | |
|---|---|---|---|---|---|---|---|---|
| | | | AAV | Age at | Time after | | | |
| | | | Treatment | AAV | vector | | | |
| Group | Stain | N | (AAVT) | injection | injection | | Collections | Analysis |
| 1 | C57BL/6 | 8 M/F | AAT-eGFP | 2 Weeks | 3 | Days | Term | Liver: |
| 2 | | 9 M/F | AAT-eGFP | 2 Weeks | 4 | Weeks | Liver | Viral genomes |
| 3 | | 7 M/F | AAT-eGFP- | 2 Weeks | 3 | Days | | and |
| | | | IFN-β-For | | | | | histological |
| 4 | | 6 M/F | AAT-eGFP- | 2 Weeks | 4 | Weeks | | examination |
| | | | IFN-β-For | | | | | (IHC): % of |
| 5 | | 7 M | AAT-eGFP | 3 Weeks | 3 | Days | | GFP positive |
| 6 | | 6 M/2 F | AAT-eGFP | 3 Weeks | 3 | Weeks | | hepatocytes. |
| 4 | | 4 M/2 F | AAT-eGFP- | 3 Weeks | 3 | Days | | |
| | | | IFN-β-For | | | | | |
| 5 | | 4 M/3 F | AAT-eGFP- | 3 Weeks | 3 | Weeks | | |
| | | | IPN-β-For | | | | | |

(N means “Number”, «AAV-AAT-eGFP-IFN-β-For » refers to a vector according to the invention, and « AAV-AAT-eGFP » refers to the same vector but without the S/MAR sequences).

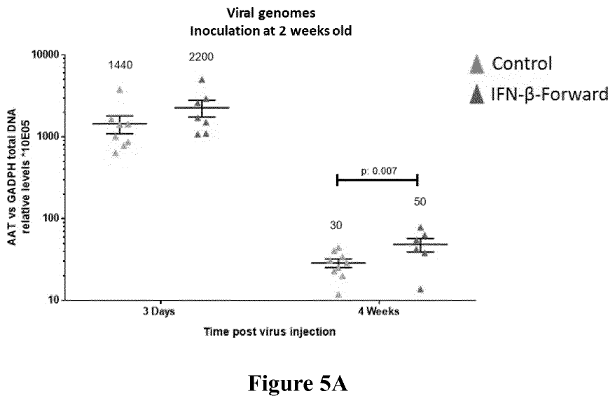

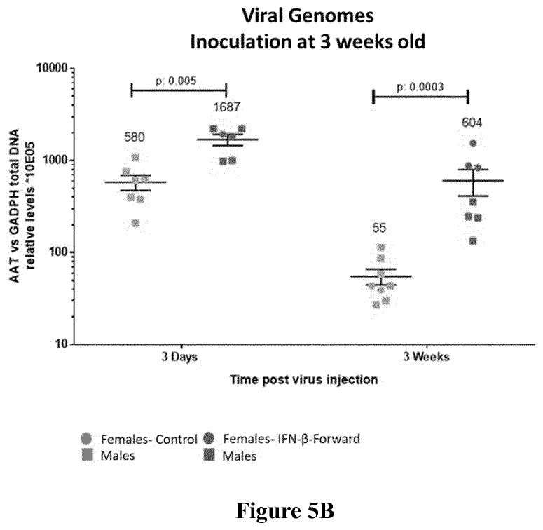

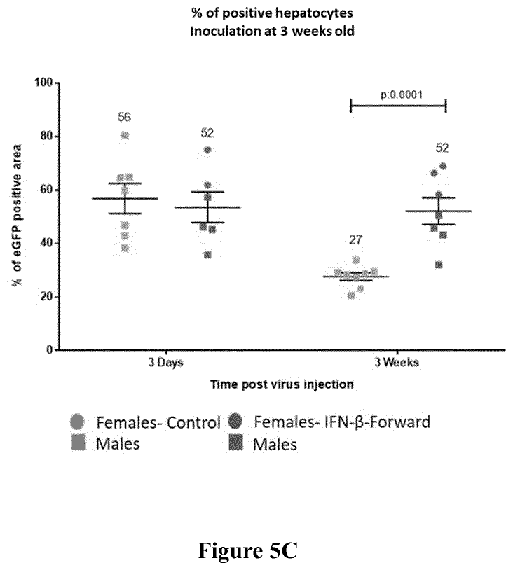

When mice are inoculated at 2-weeks old, 3 days after virus inoculation, infectivity of the different viruses was measured by the quantification of viral genomes, showing a statistically similar infectivity between the control vector, AAV-AAT-eGFP, and the S/MAR vector, AAV-AAT-eGFP-IFN-β-Forward (95% confidence level, two-tailed t test. AAV-AAT-eGFP Vs. AAV-AAT-eGFP-IFN-β-For p=0.09). 4-weeks after virus inoculation mice were sacrificed and liver samples were obtained for the quantification of viral genomes. Mice inoculated with the S/MAR vector show a significant increase of 1.7 folds compared with the vector w/o S/MAR (95% confidence level, two-tailed t test. AAV-AAT-eGFP Vs. AAV-AAT-eGFP-IFN-β-For p=0.007) (FIG. 5A).

When mice are inoculated at 3-weeks old, the S/MAR vector, AAV-AAT-eGFP-IFN-β-Forward, shows an increase of 3 folds on viral genomes just three days after virus inoculation compared with the control vector, AAV-AAT-eGFP, (95% confidence level, two-tailed t test. AAV-AAT-eGFP Vs. AAV-AAT-eGFP-IFN-β-For p=0.005). This difference on viral genomes presence increases at the end of the study, 3-weeks after virus inoculation, showing the S/MAR vector an increase of 11 folds on viral genomes compared with the control vector (95% confidence level, two-tailed t test. AAV-AAT-eGFP Vs. AAV-AAT-eGFP-IFN-β-For p=0.0003) (FIG. 5B). Furthermore, this increase in the number of viral genomes 3-weeks post-infection is translated into an increase of 2 folds in the % of transgene (GFP) positive liver area (95% confidence level, two-tailed t test. AAV-AAT-eGFP Vs. AAV-AAT-eGFP-IFN-β-For p=0.0001) (FIG. 5C).

Therefore, mice inoculated at 2- and 3-weeks of age with the S/MAR vector show an increase on viral genomes compared to control vector at the end of the experiment. Furthermore, mice inoculated at 3-weeks old with AAV-AAT-eGFP-IFN-β-Forward show a significant increase on the % of hepatocytes expressing the transgene 3 weeks after vector inoculation.

### 5.2.2 Mid-Term Follow Up of IFN-β-Forward Vector Inoculated to 3-Weeks Old Mice

To further analyze the effect of the S/MAR element on rAAV viral genomes maintenance in a growing liver and based on the previous results obtained when virus is inoculated to 3-weeks old mice, the inventors has studied S/MAR element mid-term impact on rAAV viral genomes maintenance when it is inoculated to 3-weeks old mice.

As it is described in the Table 4 below, C57BL/6 mice (6-14 males/females) were inoculated at 3-weeks of age with 5×10¹² vg/Kg with the vectors AAT-eGFP (reference vector) and AAT-eGFP-IFN-β-Forward (S/MAR vector) and liver samples were collected after 3 and 6 weeks after virus inoculation for the quantification of viral genomes by PCR and percentage of hepatocytes expressing the transgene (GFP) by immunochemistry.

To this end, new virus batches were produced as described on section 5.1.3 with an average titer of 1×10¹³ vg/mL.

TABLE 4
In vivo study design for mid-term rAAV viral genomes maintenance study in 3 weeks old mice.

| | | | | | Termial Age: | | |
|---|---|---|---|---|---|---|---|
| | | | AAV | Age at | Time after | | |
| | | | Treatment | AAV | vector | | |
| Group | Strain | N | (AAVT) | injection | injection | Collections | Analysis |
| 1 | C57BL/6 | 6 M | AAT-eGFP | 3 Weeks | 3 Weeks | Term | Liver: |
| 2 | | 5 M/4 F | AAT-eGFP | 3 Weeks | 6 Weeks | Liver | Viral genomes |
| 3 | | 7 M/5 F | AAT-eGFP- | 3 Weeks | 3 Weeks | | and |
| | | | IFN-β-For | | | | histological |
| 4 | | 6 M/8 F | AAT-eGFP- | 3 Weeks | 6 Weeks | | examination |
| | | | IFN-β-For | | | | (IHC): % of |
| | | | | | | | GFP positive |
| | | | | | | | hepatocytes. |

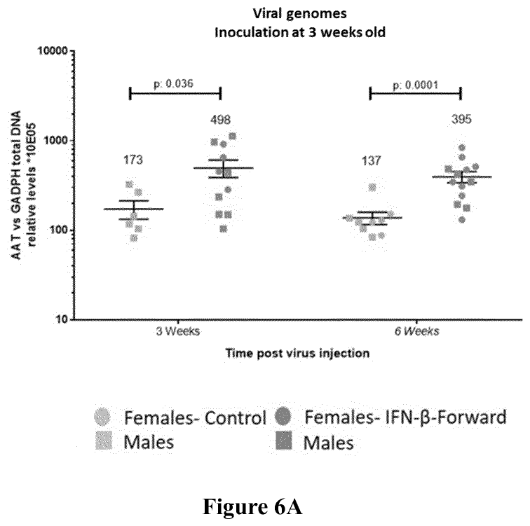

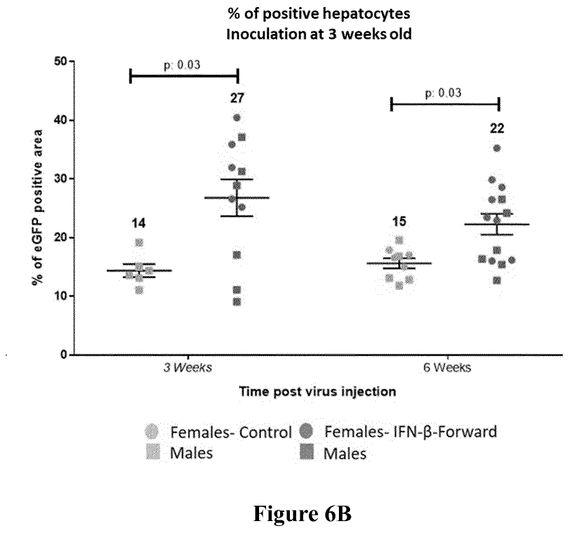

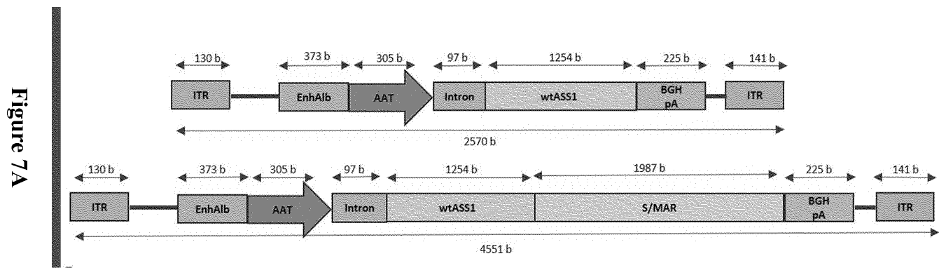

Three weeks after virus inoculation the S/MAR vector, AAV-AAT-eGFP-IFN-β-Forward, shows an increase of 3 folds on the number of viral genomes present in the liver in comparison with the control vector AAV-AAT-eGFP (95% confidence level, two-tailed t test. AAV-AAT-eGFP vs. AAV-AAT-eGFP-IFN-β-For p=0.036). Interestingly, 6 weeks after vector inoculation this difference is maintained (95% confidence level, two-tailed t test. AAV-AAT-eGFP vs. AAV-AAT-eGFP-IFN-β-For p=0.0001) (FIG. 6A). Moreover, the percentage of liver area expressing the transgene, GFP, is increased by 2 folds in the liver of mice inoculated with the S/MAR vector in comparison with the animals inoculated with the control vector (95% confidence level, two-tailed t test. AAV-AAT-eGFP vs. AAV-AAT-eGFP-IFN-β-For 3 weeks p=0.03; 6 weeks: p=0.03) (FIG. 6B).

IFN-β-Forward S/MAR element cloned into a rAAV vector is able to reduce the loss of viral genomes during liver growth when it is inoculated to 3-weeks old mice.

### 5.2.3 Effect of S/MAR Element on a Therapeutic Vector for the Treatment of CTLN1

The two previously described studies (sections 5.2.1 and 5.2.2) have demonstrated the ability of the analyzed S/MAR element, IFN-β-Forward, to maintain a higher number of rAAV genomes when vectors are inoculated to 3-weeks old mice. For this reason, the inventors have evaluated the potential of the IFN-β-Forward S/MAR sequence to increase the number of viral genomes and therapeutic gene transcription when it is included in the CTLN1 therapeutic vector and assessing it in CTLN1 mice (Fold/Fold mice).

To this end, as described in the Table 5 below, Fold/Fold mice (6-9 male/females) were inoculated at 3-weeks of age with 5×10′² vg/Kg of the reference therapeutic vector, AAV-EAlbAAT-ASS1+INT, and with the same vector presenting the IFN-β-Forward S/MAR sequence, AAV-EAlbAAT-ASS1+INT-IFN-β-Forward (S/SMAR vector). Mice were sacrificed 8 weeks after vector inoculation for liver analysis. Liver samples were collected 8 weeks after virus inoculation for the analysis of viral genomes present in the liver by PCR and the quantification of transgene expression by RT-qPCR.

TABLE 5
In vivo study design for the analysis of rAAV viral genomes maintenance
and transgene expression of the vector with the S/MAR IFN-β-Forward
sequence integrated in the CTLN1 therapeutic vector in 3 weeks old mice.

| | | | | | Terminal | | |
|---|---|---|---|---|---|---|---|
| | | | | | Age: Time | | |
| | | | AAV | Age at | after | | |
| | | | Treatment | AAV | vector | | |
| Group | Strain | N | (AAVT) | injection | injection | Collections | Analysis |
| 1 | Fold/Fold | 5 M/1 F | EAlbAAT-ASS1 + INT | 3 Weeks | 8 Weeks | Term | Liver: |
| 2 | | 5 M/4 F | EAlbAAT-ASS1 + | 3 Weeks | 8 Weeks | Liver | Viral |
| | | | INT-IFN-β-For | | | | genomes |

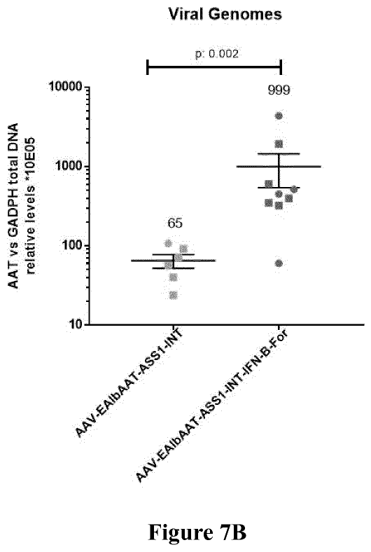

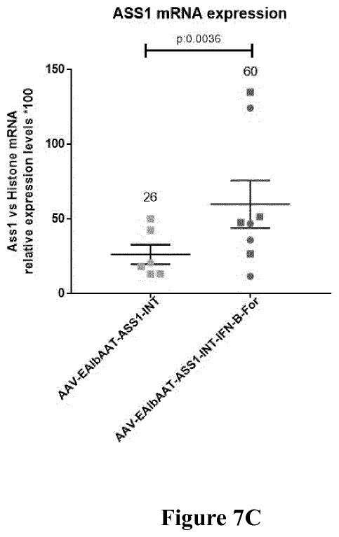

The construction of the recombinant plasmids and the production of the virus was performed as described on section 5.1.2 and 5.1.3 (FIG. 7A). The titer of the batches used for the experiment was 5×10¹² viral genomes (vg)/ml.

Mice inoculated with the S/MAR vector show an increase of 15 folds in the number of rAAV genomes present in the liver 8 weeks after virus inoculation in comparison to control vector treated mice (95% confidence level, two-tailed t test. AAV-EAlbAAT-ASS1+INT vs. AAV-EAlbAAT-ASS1+INT-IFN-β-Forward p=0.002) (FIG. 7B). Furthermore, transgene transcription is also increase by 2.5 folds in the S/MAR vector, AAV-EAlbAAT-ASS1+INT-IFN-β-Forward, treated samples in comparison with the control therapeutic vector AAV-EAlbAAT-ASS1+INT (95% confidence level, two-tailed t test. AAV-EAlbAAT-ASS1+INT vs. AAV-EAlbAAT-ASS1+INT-IFN-β-Forward p=0.003) (FIG. 7C).

All together, these results demonstrate that the IFN-β-Forward S/MAR sequence when it is included in the CTLN1 therapeutic rAAV genome provides a higher stability to the rAAV vector when it is inoculated to infantile/juvenile mice.

### CONCLUSION

The inoculation of AAV vectors with the IFN-β-Forward S/MAR sequence to 2- and 3-weeks old mice increases the number of viral genomes and the percentage of transgene positive hepatocytes (to 3 weeks old) at the end of the experiment in comparison with a control vector without any S/MAR element.

IFN-β-Forward S/MAR sequence is able to increase in the mid-term viral genomes maintenance and the percentage of cells expressing the transgene in comparison with a vector without S/MAR when it is inoculated to 3-weeks old mice.

The IFN-β-Forward S/MAR element, when it is included into the genome of a CTLN1 therapeutic vector, provides a higher stability to the rAAV genome and a stronger transgene expression when it is administered to infantile/juvenile CTLN1 mice.

### REFERENCES

Bainbridge, J. W., Mehat, M. S., Sundaram, V., Robbie, S. J., Barker, S. E., Ripamonti, C. et al. (2015). Long-term effect of gene therapy on Leber's congenital amaurosis. N. Engl. J. Med. 372, 1887-1897.
Cideciyan A V, Jacobson S G, Beltran W A, Sumaroka A, Swider M, Iwabe S, Roman A J, Olivares M B, Schwartz S B, Komáromy A M, Hauswirth W W, Aguirre G D. (2013). Human retinal gene therapy for Leber congenital amaurosis shows advancing retinal degeneration despite enduring visual improvement. Proc. Natl. Acad. Sci. USA 110: E517-E525.
Cunningham S C1, Dane A P, Spinoulas A, Logan G J, Alexander I E. (2008). Gene delivery to the juvenile mouse liver using AAV2/8 vectors. Mol Ther. 16(6): 1081-8.
Flotte T, Carter B, Conrad C, Guggino W, Reynolds T, Rosenstein B, Taylor G, Walden S, Wetzel R. (1996) A phase I study of an adeno-associated virus-CFTR gene vector in adult CF patients with mild lung disease. Hum. Gene Ther. 7: 1145-1159.
Flotte, T. R., Trapnell, B. C., Humphries, M., Carey, B., Calcedo, R., Rouhani, F. et al. (2011). Phase 2 clinical trial of a recombinant adeno-associated viral vector expressing α1-antitrypsin: interim results. Hum. Gene Ther. 22, 1239-1247.
Haase R, Argyros O, Wong S P, Harbottle R P, Lipps H J, Ogris M, Magnusson T, Vizoso Pinto M G, Haas J, Baiker A. (2010). pEPito: a significantly improved non-viral episomal expression vector for mammalian cells. BMC Biotechnol. 15; 10:20.
Hagedorn C, Schnödt-Fuchs M, Boehme P, Abdelrazik H, Lipps H J, Büning H. (2017). S/MAR Element Facilitates Episomal Long-Term Persistence of Adeno-Associated Virus Vector Genomes in Proliferating Cells. Hum Gene Ther. 28(12): 1169-1179.
Hagedorn, C., Wong, S. P., Harbottle, R., and Lipps, H. J. (2011). Scaffold/matrix attached region-based nonviral episomal vectors. Hum Gene Ther 22, 915-923.
Henry H. Q. Heng, Sandra Goetze, Christine J. Ye, Guo Liu, Joshua B. Stevens, Steven W. Bremer, Susan M. Wykes, Juergen Bode, Stephen A. Krawetz. (2004). Chromatin loops are selectively anchored using scaffold/matrix-attachment regions. J Cell Sci 117, 999-1008.
Jin C, Fotaki G, Ramachandran M, Nilsson B, Essand M, Yu D. (2016). Safe engineering of CAR T cells for adoptive cell therapy of cancer using long-term episomal gene transfer. EMBO Mol Med. 1; 8(7):702-11.
Kymäläinen H, Appelt J U, Giordano F A, Davies A F, Ogilvie C M, Ahmed S G, Laufs S, Schmidt M, Bode J, Yáñez-Muñoz R J, Dickson G. (2014) Long-term episomal transgene expression from mitotically stable integration-deficient lentiviral vectors. Hum Gene Ther. 25(5): 428-42.
Nathwani, A. C., Reiss, U. M., Tuddenham, E. G., Rosales, C., Chowdary, P., McIntosh, J. et al. (2014). Long-term safety and efficacy of factor IX gene therapy in hemophilia B. N. Engl. J. Med. 371: 1994-2004.
Nathwani, A. C., Tuddenham, E. G., Rangarajan, S., Rosales, C., McIntosh, J., Linch, D. C. et al. (2011). Adenovirus-associated virus vector-mediated gene transfer in hemophilia B. N. Engl. J. Med. 365: 2357-2365.
Piechaczek C1, Fetzer C, Baiker A, Bode J, Lipps H J. (1999). A vector based on the SV40 origin of replication and chromosomal S/MARs replicates episomally in CHO cells. Nucleic Acids Res 27: 426-428.
Schaarschmidt D, Baltin J, Stehle I M, Lipps H J, Knippers R. (2004) An episomal mammalian replicon: sequence-independent binding of the origin recognition complex. EMBO J 23:191-201.
Verghese S C, Goloviznina N A, Skinner A M, Lipps H J and Kurre P. (2014). S/MAR sequence confers long-term mitotic stability on non-integrating lentiviral vector episomes without selection. Nucleic Acids Res 42: e53.
Wang L1, Wang H, Bell P, McMenamin D, Wilson J M. (2012) Hepatic gene transfer in neonatal mice by adeno-associated virus serotype 8 vector. Hum Gene Ther. 23(5): 533-9.
Xu Z, Chen F, Zhang L, Lu J, Xu P, Liu G, Xie X, Mu W, Wang Y, Liu D. (2016). Non-integrating lentiviral vectors based on the minimal S/MAR sequence retain transgene expression in dividing cells. Sci China Life Sci. 59(10):1024-1033.

1. An expression cassette that comprises:
a transgene, and
a scaffold/matrix attachment region chosen among the nucleic acid sequences of SEQ ID NO:12 or SEQ ID NO:2, or a functional fragment or variant thereof.

2. The expression cassette according to claim 1, further comprising a promoter which initiates transgene expression upon introduction into a host cell, alpha-1-antitrypsin promoter.

3. The expression cassette according to claim 1, further comprising a 5′ITR and a 3′ITR sequences, a 5′ITR and a 3′ITR sequences of an adeno-associated virus, notably a 5′ITR and a 3′ITR sequences from the AAV2 serotype.

4. The expression cassette according to claim 1, further comprising a terminator, such as a polyadenylation signal sequence, notably the bovine growth hormone polyadenylation signal.

5. The expression cassette according to claim 1, wherein the transgene encodes argininosuccinate synthase 1.

6. The expression cassette according to claim 1, consisting of SEQ ID NO:14 or SEQ ID NO:22.

7. A recombinant vector comprising an expression cassette according to claim 1, the vector being notably an adeno-associated virus vector.

8. A host cell comprising an expression cassette according to claim 1 or the vector.

9. A viral particle comprising an expression cassette according to claim 1 or the vector.

10. The viral particle according to claim 9, which comprises capsid proteins of adeno-associated virus, notably capsid proteins from Anc80 serotype.

11. A pharmaceutical composition comprising an expression cassette according to claim 1, the vector the host cell, or the viral particle in combination with one or more pharmaceutical acceptable excipient, diluent or carrier, optionally comprising other active ingredients.

12. A method for the treatment of diseases with hepatic origin, notably citrullinemia, citrullinemia type 1 comprising the administration to a patient of a therapeutically effective amount of an expression cassette according to claim 1, the vector, the host cell, the viral particle, or the pharmaceutical composition.

13. The process for producing viral particles according to claim 9, comprising the steps of:
a) culturing a host cell according to claim 8 in a culture medium, and
b) harvesting the viral particles from the cell culture supernatant and/or inside the cells.

14. (canceled)

15. A kit comprising an expression cassette according to claim 1, the vector the host cell, or the viral particle, or the pharmaceutical composition in one or more containers, further comprising instructions or packaging materials.


SEQUENCE LISTING (23 sequences)

SEQ ID NO: 1 - DNA, 305 bp, Artificial sequence
  eGFP
ggtgccaccccctccaccttggacacaggacgctgtggtttctgagccaggtacaatgactcctttcggtaagtgcagtggaagctgtacactgcccaggcaaagcgtccgggcagcgtaggcgggactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 2 - DNA, 1989 bp, Homo sapiens
agatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatttgatagaattc

SEQ ID NO: 3 - DNA, 2355 bp, Homo sapiens
tgtgaagagacaccatgaccacagaaactcttataaaggaaagcaattattgggtccagcttacagttcagaggtttaatccattgtcatgattgcaggaagtatggtgcgccacaggcagacatggtgctggagaagtagatgagagttctatatcagattgacacacttcttccaacaaggccacacctccactcactctgagcctatggggccattttcattcaaaccaccaaagctacaaggtagcttataccccagcttgctatttctgatgagacttagtaaatagtcttaaaagcccataaaatgactcaaaactagtttttttattattattattagttcaaattaggaagaagcttgctttacatgtcaatcccttctccctctccctcatcaaaactagttttttgttttttaggttttttttcaagacagggtttctctgtgtagctttggagcctatcctggcactcgctctggagaccaggctggcctcgaactcacagagatctgcctgcctttgcctcccgagtcctgggattaaaggcatgcaccaccaacacctggccaaaattagttttaagttccagttctaggagcctccaatgccctcttttggcttccatgggaaccaggaacactatatatatatatatatatatatatatatatatatatatatatatattcaggcaaatatttatgcatataaaaataaaataaatcttttttccttttttttttaaagaagtgcaattgtcttggaatttttgtggctgctctgcccttatgtgtaactggacactaccagcatctaaacactggcctgaaaccagccaaagaaaacctttgtgccaggtcctgtgtcaaagtattatgttccttttaggatatcctatatcgtaaaggatttattttactgatagcatcttaacttcctttgaaaggttggtcttctcaagcagtcctcgtggagctggctcctcagctaatgccaggggacaataatgatcccctcccaaaaccaaacagaaaaccatggcaactctggtttccttgggcagcacctgctttaagaatgagcaaatgaccaatcagctcatgaaactaaatactctattattactaaaatatttttttgagacagggcatggaattcatcacatagttcaggttggccttgaactcagagagactcacttacctttgcctcccacgtgctggaattaaaggcatgaaccaccacaccaaacataacacttgaattttggaagagtccttcttccaatagatttgaggttttgaaaatgtggcacagaaaatatgaattcaaatataatgaaaacaagagataactttcaactaagtttctataggttcttgctaggaatcctaagcttgtctgaaactctagagcttctgtttctagtcttctgagtgttagtattgtaggtatgtgccctgcctcagtgtgatgtttttgataatcttaaagaaatcaaagaaattttataaaagactagactgtgctacacaaaaagaatattcagatgccaagaaagagttcttagaaattaagaaatatgctactagtataaatcctttataaagtggaatgacaaatctgatgaaatcttactaaaagtagaaaaacataaacatcaaagacatgaataataagaaaatcatattgtgcatatcattaacctaaaacattaacttgcaaaaatagaatagtccaaaaagtaaacaaaataaataaatcaccaagacatgatacaaggacaattcctagaatgataaaacaagaatattcattataaaaggccctatcactaaagcacaacagaaacagactcaaaagataaatcttcattgtcactggagagaagtcatactatcatagcactcagaaggaaataaaaatcaaaatgtcaaaaaggacctcagcctctgaaacacaaatacaaaatatgtccgccttcttgacaggcattactcttcaattaacattttaagaaaactataaagagagcttagtattttaagaaatctgtagctatttcttttataagcatgacaactaagtttcctgatttaaacagacctaaaaaaccggtgaagtgagtggagaaaggggatacgaagacagcatcccacatgactgctcccagtaaaggcaaggtcttcatccattttatcctgaactctgggaaatttataaagaacagaaatgtatttctctcagttctggagcctcagtccaggacactaagtctaggtactacactctcacatggtggaaactagaaagcaagctcacttgtcactcactacctgatgcctctt

SEQ ID NO: 4 - DNA, 497 bp, Homo sapiens
gaattcaaaaagatgaggtaattgtgtttttataattaaatattttataattaaaatatttataattaaaatatttataattaaatattttataattaaaatatttataattaaatattttataattaaaatatttataattaaatattttataattaaaatgtttataattaaatattttataattaaaatgtttataattacatattttataattaaaatgtttataattacatattttataattaaaatgtttataattacatattttataattacatattttataaagtatttataattacatattttataattaaagtatttataattacatattttataattaaagtatttataattacatattttataattcaatattttataaatagttaaaaagacgaggaaaaaattaaaaagacgaggttattgatctcaggaattgtatttgccaagtgagaaggaaaaaatattcacaaaggcttgtagaattc

SEQ ID NO: 5 - DNA, 1238 bp, Homo sapiens
gaattcggatcccattctccttgatgtactaatttttctttaaaagtgataataatagctcccatttagaatttttaaataacacaacaaatgtaaagtaactaatgtgtcctctggatcatggtaagtaatgaataaatttaactccctttaccttctccctttgctattttttccatgctaggatttatacatttttaaaaaactaaatctgctatcaaatgacagctttaaatttactttttaaaatttgttattgtatatatttatggggtataaagtgatgttatgatatatatatacacaatgtacactgattaaatcaagccaattaacattttatcatctcaaatacttaacattttttgtagtgagaacatttgaaatttacttttagcaatttcaaaacatacattattattattaactatagtcaccatgatgtaccatagatctttaaaaacttattcttcctgcctaactgaaactttgtactctttgactaacatcttttcattcccccacttcccagcctctggtaatcaccattacacactctgcttctatgagttcaattgctttagactccacgtaataaatgagatcatgcagcatttggctttctgtgctggcttatccttgcttagcatggtgtcttacaggttcatccatgttgcaacaaataacagaatctcattctttgttaaggctgaatactattccattgggtatatataccacattttccttatccattaatccactgatggacccttaggttgttgattccatatattggctattgtaaatagtgcagcaatgaacatgagagtgcaactatctcttcaatgtactgatttcgaatccttcggatctatctcagaagtgagattgcaggatcatataattctacttttagtcttttgaggagctccatacagctttccatatggccatactaattacattctcatcaacagtgtacaatggtttccttttctccacatcctcaccaacatttataattttttgtctttttgataatagccatctgacaggtgtaaagtgatagctcattgcagttttaatttgcattttttgatgattagtaatgttgagaattttttcatatatctcttggccagttgcatgtcttctttggaaaaatgtctattcagttcctttgcccattttttaattgggatttttggtttcttgctattgagttgtttgaattc

SEQ ID NO: 6 - DNA, 1408 bp, Homo sapiens
gaattccgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggcggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattc

SEQ ID NO: 7 - DNA, 1982 bp, Homo sapiens
gaattcggagggggatcccggcgcgccaccctccccggcaacaacctgttgccatgtatggcgatttgtatcagtcacaagcacacaacccctgctagtattaatggtgtttaaaacgttctacacgtacggcggaccgcatccgtcgcaagcacgcgcatataacccccaaatgcaccatgatgagaagcacagccacgcgtcaaaaaactttaaaaacatcgttatccaatatcattaaaaaccacaccgaaatttacacaggtagcacgtcaccgtgttagtgtcacccactgtacacaaggcgtgtcgtatatgtagtataggtatttgatgaggcggaagcatatcccgcttccagcgaacggaaataagaatcatccgttccagcatttattcaaagagggcacagaggattcacattgtttagagagagtttttcttagtcaccattccatacttgggcagtattggcctacgatttgggcgacgtttcaggctggtctattctccgtccacttttccccggctattctgtcccagcataggctcttgaaataaacaatgtttaccgagtaaaaggttccactcaccctcatttgtcgttgcacccatcccccctttgcttaatcacccgaaaactagaggacacggatggaaaacatatcgcacgcgggttgtttgaaagtcaacagctacttgtttttaatgaggacagatttgggcacaggccagagggtaaagccctacgtgtgcgcgggggggggggtgtatacgctgcgaaaacctgcacggtgcataacacccagggcgtcacgtcacatatctctgtgcacccaagtggttgttcaaccgttgttttttggatgatttttccgcaccggcttttttgtgggcgcgcataggtcggtacgcgctgtccccctaagtcccgcacggtcgttcgggcccccgtccggctcgtctccggatgaaccgtcacgttctttgtctccagaggcgacgtctccttcagatgactcgtccgtgggctcctcgtccgtcccgcccgcgggtccgacaaggaccgtcaattcgatgttatcttcgttcgcggttggccggcgcggccgtcggtatggcagtacggtcacccgggtgttatttgccgcgtataatgccctcacagtgccacttacgcggcatatgccgccaaatgcaaacacaataaatatttggtaaaacccaaagaagcagagaaaaccgagcacggccccgggggagaatgttcccgcaggagcagttaggatgaccaggagcgtccaggtgcacaacgccacgccgacaagcccagccaccaccacagacatcagcagaaacagttcaaaaatttcttggcgctccatctccggccacaggttaaggcgactacgccactgcgtgcgcgtgcggtatataacgcgacacatttgacaggccgtgtttcgagacactgttagccaagtgcttaaacactgcgggtggacgacatccagctctccggtacaggcgcaggggtgtatgccctcgttccccacctcttccctacatatccagcagatgggtccctctacaccctcttctacgtccttagacgccatctctgcagctggggtggaagtctgaaaaagggaaaggggaggtgagcagagtgcccagttagtctccgacccgccgtccgccctactgtcgctatcccgccttgacagatgtctaacgtattcacggacgccacatgtgtgtctattttcctacatccaggctttccctggaaaactgtcacaacccaccctgctttagctctacatctgtatttttgtttacgcacaggatcaacgcttcgtgcccgtccacccccgcgctctccgcctgtgtttggaggttttatgagtggttagttctaggcagctccggacaagttgtccaaaacacggcgcgccccgcccttccttccctccgaattc

SEQ ID NO: 8 - DNA, 1989 bp, Homo sapiens
gaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgttgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtataccttcacaatattaagtcttttagttcatgaatatagtatgtctctccgtttctgcattaacttagacattcattaatttctctcacaatttataagtttatttagatct

SEQ ID NO: 9 - DNA, 2355 bp, Homo sapiens
aagaggcatcaggtagtgagtgacaagtgagcttgctttctagtttccaccatgtgagagtgtagtacctagacttagtgtcctggactgaggctccagaactgagagaaatacatttctgttctttataaatttcccagagttcaggataaaatggatgaagaccttgcctttactgggagcagtcatgtgggatgctgtcttcgtatcccctttctccactcacttcaccggttttttaggtctgtttaaatcaggaaacttagttgtcatgcttataaaagaaatagctacagatttcttaaaatactaagctctctttatagttttcttaaaatgttaattgaagagtaatgcctgtcaagaaggcggacatattttgtatttgtgtttcagaggctgaggtcctttttgacattttgatttttatttccttctgagtgctatgatagtatgacttctctccagtgacaatgaagatttatcttttgagtctgtttctgttgtgctttagtgatagggccttttataatgaatattcttgttttatcattctaggaattgtccttgtatcatgtcttggtgatttatttattttgtttactttttggactattctatttttgcaagttaatgttttaggttaatgatatgcacaatatgattttcttattattcatgtctttgatgtttatgtttttctacttttagtaagatttcatcagatttgtcattccactttataaaggatttatactagtagcatatttcttaatttctaagaactctttcttggcatctgaatattctttttgtgtagcacagtctagtcttttataaaatttctttgatttctttaagattatcaaaaacatcacactgaggcagggcacatacctacaatactaacactcagaagactagaaacagaagctctagagtttcagacaagcttaggattcctagcaagaacctatagaaacttagttgaaagttatctcttgttttcattatatttgaattcatattttctgtgccacattttcaaaacctcaaatctattggaagaaggactcttccaaaattcaagtgttatgtttggtgtggtggttcatgcctttaattccagcacgtgggaggcaaaggtaagtgagtctctctgagttcaaggccaacctgaactatgtgatgaattccatgccctgtctcaaaaaaatattttagtaataatagagtatttagtttcatgagctgattggtcatttgctcattcttaaagcaggtgctgcccaaggaaaccagagttgccatggttttctgtttggttttgggaggggatcattattgtcccctggcattagctgaggagccagctccacgaggactgcttgagaagaccaacctttcaaaggaagttaagatgctatcagtaaaataaatcctttacgatataggatatcctaaaaggaacataatactttgacacaggacctggcacaaaggttttctttggctggtttcaggccagtgtttagatgctggtagtgtccagttacacataagggcagagcagccacaaaaattccaagacaattgcacttctttaaaaaaaaaaggaaaaaagatttattttatttttatatgcataaatatttgcctgaatatatatatatatatatatatatatatatatatatatatatatatagtgttcctggttcccatggaagccaaaagagggcattggaggctcctagaactggaacttaaaactaattttggccaggtgttggtggtgcatgcctttaatcccaggactcgggaggcaaaggcaggcagatctctgtgagttcgaggccagcctggtctccagagcgagtgccaggataggctccaaagctacacagagaaaccctgtcttgaaaaaaaacctaaaaaacaaaaaactagttttgatgagggagagggagaagggattgacatgtaaagcaagcttcttcctaatttgaactaataataataataaaaaaactagttttgagtcattttatgggcttttaagactatttactaagtctcatcagaaatagcaagctggggtataagctaccttgtagctttggtggtttgaatgaaaatggccccataggctcagagtgagtggaggtgtggccttgttggaagaagtgtgtcaatctgatatagaactctcatctacttctccagcaccatgtctgcctgtggcgcaccatacttcctgcaatcatgacaatggattaaacctctgaactgtaagctggacccaataattgctttcctttataagagtttctgtggtcatggtgtctcttcaca

SEQ ID NO: 10 - DNA, 497 bp, Homo sapiens
gaattctacaagcctttgtgaatattttttccttctcacttggcaaatacaattcctgagatcaataacctcgtctttttaattttttcctcgtctttttaactatttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaatattttaattataaaatatttaattataaaaacacaattacctcatctttttgaattc

SEQ ID NO: 11 - DNA, 1238 bp, Homo sapiens
gaattcaaacaactcaatagcaagaaaccaaaaatcccaattaaaaaatgggcaaaggaactgaatagacatttttccaaagaagacatgcaactggccaagagatatatgaaaaaattctcaacattactaatcatcaaaaaatgcaaattaaaactgcaatgagctatcactttacacctgtcagatggctattatcaaaaagacaaaaaattataaatgttggtgaggatgtggagaaaaggaaaccattgtacactgttgatgagaatgtaattagtatggccatatggaaagctgtatggagctcctcaaaagactaaaagtagaattatatgatcctgcaatctcacttctgagatagatccgaaggattcgaaatcagtacattgaagagatagttgcactctcatgttcattgctgcactatttacaatagccaatatatggaatcaacaacctaagggtccatcagtggattaatggataaggaaaatgtggtatatatacccaatggaatagtattcagccttaacaaagaatgagattctgttatttgttgcaacatggatgaacctgtaagacaccatgctaagcaaggataagccagcacagaaagccaaatgctgcatgatctcatttattacgtggagtctaaagcaattgaactcatagaagcagagtgtgtaatggtgattaccagaggctgggaagtgggggaatgaaaagatgttagtcaaagagtacaaagtttcagttaggcaggaagaataagtttttaaagatctatggtacatcatggtgactatagttaataataataatgtatgttttgaaattgctaaaagtaaatttcaaatgttctcactacaaaaaatgttaagtatttgagatgataaaatgttaattggcttgatttaatcagtgtacattgtgtatatatatatcataacatcactttataccccataaatatatacaataacaaattttaaaaagtaaatttaaagctgtcatttgatagcagatttagttttttaaaaatgtataaatcctagcatggaaaaaatagcaaagggagaaggtaaagggagttaaatttattcattacttaccatgatccagaggacacattagttactttacatttgttgtgttatttaaaaattctaaatgggagctattattatcacttttaaagaaaaattagtacatcaaggagaatgggatccgaattc

SEQ ID NO: 12 - DNA, 1408 bp, Homo sapiens
gaattcctgggtttggagtgagcaggggactggcactggttaactaaagtaacaggagtttcttcctcaccttcctcccaactccccagtccttatgcctctttgtgtacttaactgctacctttgacctcttgtatctcctttgcctacctctcaccttctcacctgccttctgccattccttctaactgtcaactctggctgttatcatagtctccaggaagcatcttagaacctccaagaacttaaagaccttaaggcccccagacccatttcaacagaggaaaactcttgcctttcttaagaccgccctctgaaggggcaattgatgaaaacaaacagggatggtggtggttgggagggggaagaaacgaaacaaaacattcacaacttaagatttggctcaatgatatatttgccagttattttattttttctaaaaacaatagaaaaaaatcaactttaaaaagcaaaatgtacttaaataaaaaaaattagggtttatagtacctataatactaggtactatatactaggattgaaattctgtgtaactgctataaacgttttattaaagttatttacatttaatggcaatatttacagagaaacattgtgtaaatcttaaaattttttaaaaacaattcttaaatacaaatctgttaaagaaaaaaaaaagatggtaagcataaaaaagttcttttatgcccaaagtccaatttgaggcagtttacattatggctaaatctttcagtctcaagactcagccaaggttgtgaggttgcatttgatcatgcatttgaaacaagttcataggtgattgctcaggacatttctgttagaaggaatcgttttccttacttttccttacgcacaagagttccgtagctgttcaagtttgtgtttcaactgttctcgtcgtttccgcaacaagtcctcttcagaaatgagcttttgctcctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgaggacctggggctggtgcattttcggttgttgctgatctgtctcaggactctgacactgtccaacttgaccctcttggcagcaggatagtccttccgagtggagggaggcgctgcgtagttgtgctgatgtgtggagacgtggcacctcttgaggaccagtgggctgtgaggaggtttgctgtggcctccagcagaaggtgatccagactctgaccttttgccaggagcctgcctcttttccacagaaacaacatcggaattc

SEQ ID NO: 13 - DNA, 1982 bp, Homo sapiens
gaattcggagggaaggaagggcggggcgcgccgtgttttggacaacttgtccggagctgcctagaactaaccactcataaaacctccaaacacaggcggagagcgcgggggtggacgggcacgaagcgttgatcctgtgcgtaaacaaaaatacagatgtagagctaaagcagggtgggttgtgacagttttccagggaaagcctggatgtaggaaaatagacacacatgtggcgtccgtgaatacgttagacatctgtcaaggcgggatagcgacagtagggcggacggcgggtcggagactaactgggcactctgctcacctcccctttccctttttcagacttccaccccagctgcagagatggcgtctaaggacgtagaagagggtgtagagggacccatctgctggatatgtagggaagaggtggggaacgagggcatacacccctgcgcctgtaccggagagctggatgtcgtccacccgcagtgtttaagcacttggctaacagtgtctcgaaacacggcctgtcaaatgtgtcgcgttatataccgcacgcgcacgcagtggcgtagtcgccttaacctgtggccggagatggagcgccaagaaatttttgaactgtttctgctgatgtctgtggtggtggctgggcttgtcggcgtggcgttgtgcacctggacgctcctggtcatcctaactgctcctgcgggaacattctcccccggggccgtgctcggttttctctgcttctttgggttttaccaaatatttattgtgtttgcatttggcggcatatgccgcgtaagtggcactgtgagggcattatacgcggcaaataacacccgggtgaccgtactgccataccgacggccgcgccggccaaccgcgaacgaagataacatcgaattgacggtccttgtcggacccgcgggcgggacggacgaggagcccacggacgagtcatctgaaggagacgtcgcctctggagacaaagaacgtgacggttcatccggagacgagccggacgggggcccgaacgaccgtgcgggacttagggggacagcgcgtaccgacctatgcgcgcccacaaaaaagccggtgcggaaaaatcatccaaaaaacaacggttgaacaaccacttgggtgcacagagatatgtgacgtgacgccctgggtgttatgcaccgtgcaggttttcgcagcgtatacacccccccccccgcgcacacgtagggctttaccctctggcctgtgcccaaatctgtcctcattaaaaacaagtagctgttgactttcaaacaacccgcgtgcgatatgttttccatccgtgtcctctagttttcgggtgattaagcaaaggggggatgggtgcaacgacaaatgagggtgagtggaaccttttactcggtaaacattgtttatttcaagagcctatgctgggacagaatagccggggaaaagtggacggagaatagaccagcctgaaacgtcgcccaaatcgtaggccaatactgcccaagtatggaatggtgactaagaaaaactctctctaaacaatgtgaatcctctgtgccctctttgaataaatgctggaacggatgattcttatttccgttcgctggaagcgggatatgcttccgcctcatcaaatacctatactacatatacgacacgccttgtgtacagtgggtgacactaacacggtgacgtgctacctgtgtaaatttcggtgtggtttttaatgatattggataacgatgtttttaaagttttttgacgcgtggctgtgcttctcatcatggtgcatttgggggttatatgcgcgtgcttgcgacggatgcggtccgccgtacgtgtagaacgttttaaacaccattaatactagcaggggttgtgtgcttgtgactgatacaaatcgccatacatggcaacaggttgttgccggggagggtggcgcgccgggatccccctccgaattc

SEQ ID NO: 14 - DNA, 3610 bp, Artificial sequence
  vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgatctacaggtgccaccccctccaccttggacacaggacgctgtggtttctgagccaggtacaatgactcctttcggtaagtgcagtggaagctgtacactgcccaggcaaagcgtccgggcagcgtaggcgggactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaaggtacctcgccaccggatccaagaggtaagggtttaagggatggttggttggtggggtattaatgtttaattacctgttttacaggcctgaaatcacttggttttaggttgggccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagcgaagcggccgcgactctaggaattcctgggtttggagtgagcaggggactggcactggttaactaaagtaacaggagtttcttcctcaccttcctcccaactccccagtccttatgcctctttgtgtacttaactgctacctttgacctcttgtatctcctttgcctacctctcaccttctcacctgccttctgccattccttctaactgtcaactctggctgttatcatagtctccaggaagcatcttagaacctccaagaacttaaagaccttaaggcccccagacccatttcaacagaggaaaactcttgcctttcttaagaccgccctctgaaggggcaattgatgaaaacaaacagggatggtggtggttgggagggggaagaaacgaaacaaaacattcacaacttaagatttggctcaatgatatatttgccagttattttattttttctaaaaacaatagaaaaaaatcaactttaaaaagcaaaatgtacttaaataaaaaaaattagggtttatagtacctataatactaggtactatatactaggattgaaattctgtgtaactgctataaacgttttattaaagttatttacatttaatggcaatatttacagagaaacattgtgtaaatcttaaaattttttaaaaacaattcttaaatacaaatctgttaaagaaaaaaaaaagatggtaagcataaaaaagttcttttatgcccaaagtccaatttgaggcagtttacattatggctaaatctttcagtctcaagactcagccaaggttgtgaggttgcatttgatcatgcatttgaaacaagttcataggtgattgctcaggacatttctgttagaaggaatcgttttccttacttttccttacgcacaagagttccgtagctgttcaagtttgtgtttcaactgttctcgtcgtttccgcaacaagtcctcttcagaaatgagcttttgctcctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgaggacctggggctggtgcattttcggttgttgctgatctgtctcaggactctgacactgtccaacttgaccctcttggcagcaggatagtccttccgagtggagggaggcgctgcgtagttgtgctgatgtgtggagacgtggcacctcttgaggaccagtgggctgtgaggaggtttgctgtggcctccagcagaaggtgatccagactctgaccttttgccaggagcctgcctcttttccacagaaacaacatcggaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 15 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 16 - DNA, 141 bp, Artificial sequence
  5′ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 141 bp, Artificial sequence
  3′ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 225 bp, Artificial sequence
  BGHpA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 19 - DNA, 305 bp, Artificial sequence; identical to SEQ ID NO: 1
  AAT promoter

SEQ ID NO: 20 - DNA, 1242 bp, Homo sapiens
atgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagcga

SEQ ID NO: 21 - DNA, 104 bp, Artificial sequence
  MVM intron
ggatccaagaggtaagggtttaagggatggttggttggtggggtattaatgtttaattacctgttttacaggcctgaaatcacttggttttaggttgggccacc

SEQ ID NO: 22 - DNA, 4191 bp, Artificial sequence
  vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgatctacaggtgccaccccctccaccttggacacaggacgctgtggtttctgagccaggtacaatgactcctttcggtaagtgcagtggaagctgtacactgcccaggcaaagcgtccgggcagcgtaggcgggactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaaggtacctcgccaccggatccaagaggtaagggtttaagggatggttggttggtggggtattaatgtttaattacctgttttacaggcctgaaatcacttggttttaggttgggccaccatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagcgaagcggccgcgactctagagatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatttgatagaattcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 23 - DNA, 130 bp, Artificial sequence
  5′ITR
cctgcaggcagctgcgcgctcgctcgctcactgaggccgcccgggcgtcgggcgacctttggtcgcccggcctcagtgagcgagcgagcgcgcagagagggagtggccaactccatcactaggggttcct